(19) United States
(12) Patent Application Publication
(10) Pub. No.: US 2024/0034985 A1
(43) Pub. Date: Feb. 1, 2024

(54) COMPOSITIONS FROM A BACTERIAL ORGANISM AND USES THEREOF

(71) Applicant: LIBERTY BIOSECURITY, LLC, Worcester, MA (US)

(72) Inventors: Kyle S. LANDRY, Worcester, MA (US); Michaele MCGREGOR, Worcester, MA (US)

(21) Appl. No.: 18/265,665

(22) PCT Filed: Dec. 17, 2021
(86) PCT No.: PCT/US21/64059

§ 371 Date: Jun. 6, 2023

Related U.S. Application Data
(60) Provisional application No. 63/127,216, filed on Dec. 18, 2020.

(51) Int. Cl. C12N 1/20; C12N 1/06; A61Q 17/04; A61Q 19/08; A61K 8/99; A61K 8/49; A61K 8/34; C09D 7/48

(52) U.S. Cl. CPC C12N 1/205; C12N 1/066; A61Q 17/04; A61Q 19/08; A61K 8/99; A61K 8/498; A61K 8/347; C09D 7/48; C12R 2001/07

(57) ABSTRACT

The technology relates in part to a strain of Bacillus pumilus bacteria deposited under ATCC accession number PTA-126909 and compositions derived from the bacteria for a variety of uses, including cosmetic and commercial compositions. The bacteria may be processed in a variety of ways, such as disrupting the bacteria (e.g., physically disrupting and lysing bacteria by lyophilizing aqueous batches of the bacteria, microfluidization of batches of bacteria, and the like), with the cellular remains and/or cellular secretions from such processing being then formulated with additional components to make useful end products, such as topical sunblock, sunscreen compositions with SPF-boosting properties, or household paint. In various embodiments, the compositions derived from the bacteria may be applied to surfaces to provide protection from ultraviolet light, or embedded within compositions to provide UV stability.

### RELATED APPLICATION

This application claims priority to U.S. Provisional Application 63/127,216, filed Dec. 18, 2020, naming Kyle Landry et al. as inventors, entitled “COMPOSITIONS FROM A BACTERIAL ORGANISM AND USES THEREOF,” which is incorporated herein by reference in its entirety for all purposes.

### FIELD

The technology relates in part to cosmetic and other compositions derived from bacteria that afford protection against ultraviolet light. The technology relates in part to a strain of Bacillus pumilus bacteria deposited under ATCC accession number PTA-126909 and compositions derived from the bacteria for a variety of uses.

### BACKGROUND

Several physiologically and phylogenetically distinct microorganisms have been encountered while examining microbial contamination of surfaces. Some of these microorganisms form round, exosporium-bearing spores, whose exosporia might be responsible for adaptation to the extreme conditions of, and direct adhesion to, surfaces with exposure to ultraviolet light.

### SUMMARY

Isolation, identification and understanding of ultraviolet (UV)-resistant microorganisms can be of significant use in industry and in medicine. For example, material from cellular secretions, cellular debris from lysis, and/or exosporium components (e.g., proteins, lipids, etc. that are isolated and optionally purified) can be used to manufacture UV-resistant compositions and articles. In non-limiting examples, a composition comprising a microorganism and/or cellular component described herein can be utilized in cosmetics or sunscreens or to prolong the product life of articles (e.g., convertible tops, tents). A composition comprising such a microorganism and/or cellular component thereof can be applied to an article (e.g., as an UV-retardant spray or UV-blocking topical composition or SPF-boosting topical composition for biological surfaces) or can be incorporated into an article (e.g., as a component in exterior paints).

Provided in certain aspects are highly UV-resistant bacterial isolates useful for manufacturing new and improved UV-resistant compositions. Provided in certain aspects is an isolated, and optionally a biologically purified, culture of a novel spore forming Bacillus species, referred to herein as “an isolate.” In particular aspects, provided is a Bacillus pumilus isolate with high UV-resistant properties, having ATCC accession number PTA-126909.

Additionally, because of its UV-resistant properties, a component or a mixture of components from a B. pumilus strain (e.g., a protein, lipid, etc. that is isolated and optionally purified), optionally treated to increase UV resistance, can be used to manufacture a UV-resistant product. In non-limiting examples, one or more components from such a strain could be incorporated in a topical composition such as a sunscreen, applied to a biological surface to provide UV-protection or to a non-biological substrate to prolong the structural integrity of the substrate exposed to UV light (e.g., convertible top, tent, painted surface; e.g., applied as a UV-retardant spray), or integrated into a composition or substrate to prolong the structural integrity of the substrate or reduce UV transmission (e.g., incorporation in a paint, plastic or glass). Additionally, one or more components or a complex mixture from such a strain could be used to impart desirable properties in a topical or cosmetic composition, such as anti-aging properties or antioxidant properties.

### BRIEF DESCRIPTION OF THE DRAWINGS

FIG. 1 provides the UV spectra for a test composition (Trace A) versus a comparative sample (oxybenzone), where the Y-axis is UV absorbance (scale of 0-4) and the X-axis is wavelength (200-400 nm), as described in Example 2. Trace A is a test composition at 1.0 mg/mL; and Trace B is oxybenzone at 0.1 mg/mL.

FIG. 2 shows solar spectral irradiance according to international standard ISO 9845-1 (First edition 1992 Oct. 15) (upper trace) vs. solar spectral irradiance according to international standard ISO 9845-1 transmitted through the test product (i.e., Bacillus Lysate described herein; lower trace).

FIG. 3 shows results of an MTT assay with the values presented as the mean percent viability±the standard deviation of the mean.

FIG. 4 shows results of an ELISA assay for collagen. The values are presented as mean concentration (ng/ml)±the standard deviation of the mean.

FIG. 5 shows results of an ELISA assay for hyaluronic acid. The values are presented as mean concentration (ng/ml)±the standard deviation of the mean.

FIG. 6 shows results of an ELISA assay for elastin. The values are presented as mean concentration (ng/ml)±the standard deviation of the mean.

FIG. 7 shows results of a HORAC assay (upper graph and upper table: positive control; lower graph and lower table: test material (i.e., Bacillus Lysate described herein)).

FIG. 8 shows results of an ORAC assay (upper graph and upper table: positive control; lower graph and lower table: test material (i.e., Bacillus Lysate described herein)).

### DETAILED DESCRIPTION

### Definitions

Unless defined otherwise, all technical and scientific terms used herein have the meaning commonly understood by a person skilled in the art of the present disclosure. As used herein, the following terms have the meanings ascribed to them below, unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about. Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

The terms “optional” or “optionally” as used herein means that a subsequently described event or circumstance may but need not occur, and that the description includes instances where the event or circumstance occurs and instances in which it does not. For example, a component that is “optionally purified” means that the component may be purified or that the component may not be purified.

The term “purified,” as described herein, refers to the purity of a given compound. For example, a compound is “purified” when the given compound is a major component of the composition, i.e., at least about 50% w/w pure. Thus, “purified” embraces at least about 50% w/w purity, at least about 60% w/w purity, at least about 70% purity, at least about 80% purity, at least about 85% purity, at least about 90% purity, at least about 92% purity, at least about 94% purity, at least about 96% purity, at least about 97% purity, at least about 98% purity, at least about 99% purity, at least about 99.5% purity, and at least about 99.9% purity, wherein “substantially pure” embraces at least about 97% purity, at least about 98% purity, at least about 99% purity, at least about 99.5% purity, and at least about 99.9% purity.

The term “isolated,” as described herein, refers to a component or organism that has been separated from its original environment. An organism can be separated from a natural geographic environment and optionally can exist in an environment different than the original natural geographic environment (e.g., in a container such as a laboratory container (e.g., cell culture container) or storage container), for example. A component of an organism can be separated from the organism in purified form or non-purified form and exist in an ex vivo environment (e.g., in a form in which cells have been disrupted and/or in a container), for example.

The phrase “essentially free of viable cells” means that there are no CFUs or colony forming units upon visual inspection following seeding of cellular material onto an appropriate semisolid growth medium for an appropriate period of time (e.g., 24 hours) that would otherwise produce a population of cells visible to the naked eye.

### Biological Deposit

The bacterial strain disclosed in this description has been deposited under conditions that assure that access to the cultures will be available during the pendency of this application. The bacterial strain disclosed in this description has been deposited in the American Type Culture Collection (ATCC), 10801 University Blvd., Manassas, Va. 20110, USA, as PTA-126909. The deposit was received by the ATCC on Dec. 9, 2020 and was given an accession number by the International Depository Authority of PTA-126909. The deposit has been made to and received by the International Depository Authority under the provisions of the Budapest Treaty, and all restrictions upon public access to the deposit will be irrevocably removed upon the grant of a patent on this application. The deposits will be available as required by foreign patent laws in countries wherein counterparts of the subject application, or its progeny, are filed. However, it should be understood that the availability of the deposits does not constitute a license to practice the subject invention.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of a deposit, and in any case, for a period of at least thirty (30) years after the date of deposit or for the enforceable life of any patent which may issue disclosing the cultures. The depositor acknowledges the duty to replace the deposit(s) should the depository be unable to furnish a sample when requested due to the condition of the deposit(s).

Compounds and Compositions, including Compositions for Use as Additives

In various embodiments, provided is an isolated biologically pure culture of a strain of Bacillus pumilus deposited under ATCC accession number PTA-126909. In addition, provided herein are compositions comprising a strain of Bacillus pumilus deposited under ATCC accession number PTA-126909. The cells of the culture may be in any form, for example as vegetative cells, as spores, and as non-viable cells. Further provided are compositions comprising cellular remains and/or secretions from a strain of Bacillus pumilus deposited under ATCC accession number PTA-126909, wherein the cellular remains are non-viable. Such cellular remains may be lysed, secreted, lyophilized from, sonicated, microfluidized, or otherwise derived or obtained from the strain of Bacillus pumilus. Further provided are compositions comprising such cellular remains and/or secretions, wherein the cellular remains and/or secretions are designated by the INCI name “Bacillus Lysate” according to the International Cosmetic Ingredient Nomenclature Committee (INC) and the International Cosmetic Ingredient Dictionary and Handbook. Such compositions may be articles of commerce by themselves, or may be further formulated as additives in other compositions.

Without being bound by theory, it is believed that the physical makeup of the deposited bacterial strain rather than biochemical reactions (an enzymatic response to repair damage or otherwise neutralized UV radiation) is responsible for its heightened resistance to UV light. Furthermore, without being bound by theory, it is believed that multiple components derived from the bacteria, possibly including spore coats and other materials, contribute to UV absorbance.

In various embodiments, the compositions comprising the bacteria or the cellular remains of the bacteria are topical compositions for application to the human body and skin, such as cosmetics, sunscreens, or sunblocks. In various embodiments, the bacteria or its cellular remains are incorporated to provide additional sun protection factor (SPF) protection. In various embodiments, the bacteria or its cellular remains is included in a formulation with one or more chemical agents with anti-UV properties. In various embodiments, the sunscreen or sunblock composition is formulated to provide an additive or synergistic anti-UV property when formulated in combination with one or more of the following components: oxybenzone, octinoxate, octisalate, octocrylene, homosalate, and avobenzone. In various embodiments, the bacteria or its cellular remains is formulated to replace one or more chemical agents with anti-UV properties. In various embodiments, the sunscreen or sunblock composition is formulated to be essentially free of one or more of the following components: oxybenzone, octinoxate, octisalate, octocrylene, homosalate, and avobenzone. In various embodiments, the bacteria or its cellular remains and/or secretions is included in a formulation and designated by the INCI name “Bacillus Lysate” according to the International Cosmetic Ingredient Nomenclature Committee (INC) and the International Cosmetic Ingredient Dictionary and Handbook. Bacillus Lysate also is referred to as “BL” herein.

In various embodiments, the compositions according to the present disclosure are paint. By paint is meant any pigmented or non-pigmented liquid, liquefiable solid, or solid mastic that, after application to a substrate in a thin layer, converts to a solid film. The paint may be oil-based or water-based. In various embodiments, the paint is for exterior use on a house or other dwelling or residential or commercial building. The bacteria or its cellular remains can be incorporated into paint to provide additional UV protection. In various embodiments, the bacteria or its cellular remains replace one or more chemical agents with anti-UV properties.

More generally, in various embodiments, the compositions according to the present disclosure can include layered compositions comprising (i) a substrate upon which the strain of bacteria or its cellular remains are deposited on a surface of the substrate, and (ii) a surface layer of bacteria or cellular remains, optionally incorporated in a film.

In various embodiments, the compositions according to the present disclosure are solid materials, including but not limited to fabric, textile, plastic, metal, wood, paper, paperboard, and glass. Such materials can have the bacteria or its cellular remains coated, integrated, embedded, impregnated, or otherwise incorporated. Such application can reduce or prevent UV transmission through the materials, increase durability and reduce wear-and-tear on the materials from UV sources, and otherwise provide UV protection. Exemplary applications include but are not limited to windows, eyeglasses, tarps, tents, umbrellas, clothing, swim wear, footwear, draperies, window shades, outdoor furniture coverings, outdoor furniture cushions, awnings, flags, pool covers, motor vehicle covers, and boat covers. In various embodiments, the compositions are macroscopically homogeneous or macroscopically heterogeneous.

In certain instances, a composition is obtainable by a process that includes growing the bacteria in artificial ultraviolet light and thereafter exposing the bacteria to mechanical disruption. Any suitable growth conditions can be utilized, using, for example, bacterial cell growth media and containers known in the art that facilitate cell division. Any suitable artificial ultraviolet light source and duration can be utilized, and sometimes the artificial ultraviolet light is greater than or equal to 700 joules/m² of exposure (fluence), and optionally approximately 900 joules/m² of exposure (fluence), is utilized. Any suitable physical disruption process can be utilized, non-limiting examples of which include microfluidization, sonication and/or lyophilization. A composition described herein sometimes is obtainable by a process that includes isolating the bacteria after growth in the artificial ultraviolet light prior to exposing the bacterial to mechanical disruption.

In certain implementations the bacteria were exposed to ultraviolet light naturally occurring in the stratosphere of Earth or beyond the stratosphere of Earth prior to growing the bacteria in the artificial ultraviolet light. In certain instances, the bacteria were exposed to the ultraviolet light naturally occurring in the stratosphere of Earth or beyond the stratosphere of Earth for about 18 months or more. In certain implementations, the bacteria were exposed to space, for example, as described in Rabbow et al, Astrobiology, Volume 12, Number 5, pages 374-386, 2012. In certain implementations, the bacteria were derived from ATCC deposit PTA-7603, see also U.S. Pat. No. 7,262,047, the contents of which are incorporated by reference in their entirety. In certain implementations, the bacteria were derived from various strains, for example SAFR-032, UV-Space (56T-2), UV-Mars (183T-1), Dark-Space (40T-5), and Dark-Mars (168T-5), as described in Chiang A J, et al, “Alteration of proteomes in first generation cultures of Bacillus pumilus spores exposed to outer space,” mSystems 4:e00195-19, volume 4, Issue 4, pp 1-15, July/August 2019. In certain implementations, the bacteria are phenotypically distinct by visual inspection from progenitor bacteria. In certain implementations, the bacteria are screened for the presence or absence of Bacillus cereus as a contaminant and are free of or essentially free of Bacillus cereus. In certain implementations, the bacteria are screened for the presence or absence of Bacillus subtilis as a contaminant, and the resulting products are free of or essentially free of Bacillus subtilis. In certain implementations, the bacteria are screened for the presence or absence of Bacillus pumilus CX-UV strain as a contaminant, and the resulting products are free of or essentially free of Bacillus pumilus CX-UV strain. In certain implementations, bacteria from the genus Bacillus, including Bacillus subtilis and/or Bacillus pumilus CX-UV strain, are grown in a reactor vessel, and following a growth phase, the intact cellular components of the reactor vessel are separated from the aqueous growth media and water-soluble contents of the reactor vessel, including the cell culture components, and subsequent to such separation, the intact cellular components are lysed to form a composition of bacterial debris which is non-viable. In various implementations, the water-soluble contents which are separated from the intact cells contain all of or essentially all of any water-soluble compounds with a molecular weight of less than 500 Daltons that were secreted by the bacterial cells during the growth phase. In various implementations, bacteria that are capable of surviving ultraviolet light at greater than or equal to 700 joules/m2 of exposure (fluence), optionally ultraviolet light at greater than or equal to 900 joules/m2 of exposure (fluence) for at least one hour under laboratory conditions, are grown in a reactor vessel, followed by a separation step where the water-soluble contents of the reactor vessel, including secreted water soluble compounds with a molecular weight of less than 500 Daltons, are separated from the intact bacterial cells, such that the separated portion contains all of or essentially all of any water-soluble compounds with a molecular weight of less than 500 Daltons that were secreted by the bacterial cells during the growth phase, followed by lysis of the remaining bacterial cells to provide a composition comprising non-viable bacterial debris.

In various implementations, the bacteria are grown substantially in the absence of conditions allowing for fermentation. In various implementations, fermentation products are completely or substantially excluded. By ‘substantially excluded’ is meant that any fermentation products that may be present do not contribute to the bulk properties of the composition. In various implementations, fermentation products that may be present are removed along with growth media prior to isolation of a composition comprising the cellular remains and/or debris of the bacterial strain. In various embodiments, bacterial fermentation products that are soluble in water and secreted by the bacteria during a growth phase are substantially excluded from compositions as described herein. In various embodiments, bacterial fermentation products that are soluble in water and secreted by the bacteria during a growth phase and have UV blocking properties are substantially excluded from compositions as described herein. For clarity, the lysed bacteria and cellular debris from such lysis as described herein are not fermentation products. In various embodiments, bacterial fermentation products that mimic the feel of clay are entirely or substantially excluded, for example Uniclay™ fermentation products.

A composition described herein sometimes is obtainable by a process that includes concentrating the cellular remains and/or secretions into an aqueous concentrate, where the aqueous concentrate is a liquid containing a concentration of the cellular remains and/or secretions greater than the concentration of the cellular remains and/or sections in a liquid subjected to the concentrating. In various embodiments, the concentrated composition is essentially free of growth media. Any suitable concentration process can be utilized, non-limiting examples of which employ use of salt, polyethylene glycol, solvent, SDS precipitation, three-phase partitioning, dialysis, centrifugation, ultrafiltration, lyophilization, affinity chromatography, immunoprecipitation and/or increased temperature. In various embodiments, the concentrated material forms a pellet or supernatant, and may be separated from excess liquid by filtering or other physical separation. In various embodiments, the concentrated material forms a separate layer, and may be separated from less concentrated material by decanting the separated layers. In various embodiments, the concentrated material following separation from less concentrated material is combined with water or an aqueous carrier to achieve a desired weight percent of solids to liquid. A composition described herein sometimes is obtainable by a process that includes combining the cellular remains and/or secretions with one or more other components, such as liquid carriers. Non-limiting examples of components that can be combined include one or more of an aqueous component, fatty component, volatile oil, non-volatile oil, surfactant, polymer, emulsifier, ultraviolet filter, sirtuin activator, anti-oxidant, and free-radical scavenger. In various embodiments, the concentrated material is re-suspended in water or aqueous carrier at a weight percent of solids to liquids from 0.001% to 10%. In various embodiments, the concentrated material is re-suspended in water or aqueous carrier at a weight percent of solids to liquids from 0.01% to 5%, from 0.1% to 4%, from 0.5% to 3%, from 1% to 2%, or any amount in between these ranges. In various implementations, bacterial lysate components (e.g., cellular remains with or without bacterial secretions, including, for example Bacillus Lysate) can constitute about 0.001% to about 10% by weight, or about 1% to about 3% by weight, or about 0.1%, about 0.5%, about 1%, about 2%, or about 3% by weight, of a combined composition. In various embodiments, cellular secretions during a bacterial growth phase are removed with growth media prior to further processing, such as lysis, such that the product after lysis is free of or essentially free of secreted products (e.g., secreted fermentation products) from the growth phase. In various embodiments, trace amounts of cellular secretions and growth media may be present after lysis, provided that their presence does not contribute to the beneficial properties of the post-lysis composition. In various implementations, water-soluble molecules with a molecular weight less than 500 Daltons secreted by the bacteria during the bacterial growth phase are essentially removed prior to lysis of bacterial cells.

A composition provided herein sometimes is essentially free of viable cells of the bacteria. In various implementations, a composition provided herein is essentially free of growth media. In certain implementations, cellular remains and/or secretions in the composition are of a Bacillus bacteria, sometimes are of a Bacillus pumilus bacteria, and sometimes are of a Bacillus pumilus bacteria deposited under ATCC accession number PTA-126909. In certain instances, cellular remains and/or secretions are from a substantially homogeneous population of bacteria. A substantially homogeneous population generally is with respect to other bacteria and refers to bacteria in a population being substantially free of other bacteria types (e.g., where the bacteria are Bacillus bacteria, a composition is substantially free of non-Bacillus bacteria, and where the bacteria are Bacillus pumilus bacteria, the composition is substantially free of non-Bacillus pumilus bacteria). A composition having a substantially homogeneous population of bacteria sometimes is a composition in which bacteria consist essentially of a particular bacteria strain. A composition in which bacteria consist essentially of a particular bacteria strain generally is a composition in which the particular bacteria strain is about 95% or more of total bacteria in the composition, and sometimes 96% or more, 97% or more, 98% or more, 99% or more, 99.5% or more, or 99.9% or more of total bacteria in the composition.

Cosmetic and End-Use Compositions

In various embodiments, compositions according to present disclosure are used as antioxidant components in cosmetic compositions, or alternatively as compositions for topical administration with antioxidant properties, as hair volumizers, as UV protector-compositions and/or hair repair, as compositions for protection of animal skin, leather, or fur, for example with UV protector or moisturizer properties, as compositions for topical administration with moisturizer properties, in compositions for supplementing the diet and/or as new dietary ingredients in dietary supplements, as post-biotic by-products, as an ingredient with product/formulation thickener properties, as a component in compositions for topical administration with anti-aging properties, as a component in paints, stain, lacquer, varnish, glaze, ink, plastics, and other materials, as a component to provide HEV (blue light) protection, and as a component in fertilizer or plant growth compositions.

In certain implementations, provided is a composition that includes cellular remains of bacteria, or secretions of bacteria, or cellular remains and secretions of bacteria, where the composition is (i) a consumer product selected from a cosmetic composition, a sunscreen and/or sunblock composition, a sun protection factor (SPF) booster, and a topically-applied pharmaceutical composition, or (ii) an additive for use in a consumer product selected from a cosmetic composition, a sunscreen and/or sunblock composition, a sun protection factor (SPF) booster, and a topically-applied pharmaceutical composition. Sun protection factor (SPF) generally is a measure of how well a sunscreen protects against UVB rays, where a composition having a higher SPF value affords greater protection against sunburn, for example. In various implementations, bacterial secretions from the growth phase of the bacteria are separated from intact bacterial cells prior to lysis of the bacterial cells to provide cellular remains which are essentially free of growth media and secretions form the growth phase (i.e., components of cell culture). In various implementations, water-soluble molecules secreted during bacterial growth with a molecular weight of less than 500 Daltons are removed from intact bacterial cells prior to lysis.

In certain implementations, a composition provided herein includes one or more components chosen from an aqueous component, fatty component, volatile oil, non-volatile oil, surfactant, polymer, emulsifier, ultraviolet filter, sirtuin activator, anti-oxidant, and free-radical scavenger, non-limiting examples of which are described herein.

In various implementations, a composition provided herein is in the form of a cream, lotion, emulsion, oil, butter, paste, balm, stick, foam, gel, serum, ointment, mousse, powder, semi-solid formulation, spray or aerosol. In various implementations, a composition provided herein is in the form of sunscreen, sunblock, body moisturizer, facial moisturizer, hair moisturizer, make-up foundation, lipstick, lip balm, hair spray, or hair dye. In various embodiments, the composition is in the form of a solution, dispersion, suspension, emulsion, or colloid. In various embodiments, the composition is in the form of a cream, lotion, paste, oil, foam, gel, serum, powder, spray or aerosol.

Aqueous Component

A composition herein (e.g., a cosmetic composition, a sunscreen and/or sunblock composition, a topically-applied pharmaceutical composition) may comprise an aqueous component. In some embodiments, an aqueous component is present in an amount ranging from about 10% to about 99% by weight of the total weight of the composition. For example, an aqueous component may be present at about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight of the total weight of the composition. In some embodiments, an aqueous component is present in an amount ranging from about 20% to about 90% by weight, from about 50% to about 85% by weight, or from about 60% to about 75% by weight of the total weight of the composition.

In some embodiments, an aqueous component comprises water. In some embodiments, an aqueous component comprises at least one organic solvent miscible with water (at room temperature 25° C.). Organic solvents may include, for example, monoalcohols, polyols, glycol ethers, and mixtures thereof. Monoalcohols may include monoalcohols having from 2 to 6 carbon atoms (e.g., ethanol, isopropanol). Polyols may include polyols having from 2 to 20 carbon atoms, 2 to 10 carbon atoms, or 2 to 6 carbon atoms (e.g., glycerol, propylene glycol, butylene glycol, pentylene glycol, hexylene glycol, caprylylglycol, dipropylene glycol, diethylene glycol). Glycol ethers may include glycol ethers having from 3 to 16 carbon atoms (e.g., mono-, di- or tri-propylene glycol (Ci-C4)alkyl ethers, mono-, di- or tri-ethylene glycol (Ci-C4) alkyl ethers).

Fatty Component

A composition herein (e.g., a cosmetic composition, a sunscreen and/or sunblock composition, a topically-applied pharmaceutical composition) may comprise one or more fatty components. Fatty components may include oils, waxes, fatty acids, fatty alcohols, and mixtures thereof. In some embodiments, a composition herein is in the form of an emulsion (e.g., an oil-in-water emulsion), and comprises a dispersed fatty component comprising at least one oil. The term oil generally refers to any fatty substance that is in liquid form at ambient temperature (20-25° C.) and at atmospheric pressure. In certain embodiments, a composition herein is oil-free.

In some embodiments, a fatty component is present in an amount ranging from about 1% to about 30% by weight of the total weight of the composition. For example, a fatty component may be present at about 2% by weight, about 5% by weight, about 10% by weight, about 15% by weight, about 20% by weight, or about 25% by weight of the total weight of the composition. In some embodiments, a fatty component is present in an amount ranging from about 2% to about 20% by weight, or about 3% to about 15% by weight of the total weight of the composition.

An oil herein may be chosen from volatile and non-volatile oils of hydrocarbon-based, silicone or fluoro type. An oil may be of animal, vegetable, mineral or synthetic origin. The term hydrocarbon-based oil generally refers to an oil formed essentially of, or consisting of, carbon and hydrogen atoms, may include oxygen and nitrogen atoms, and generally contains no silicon or fluorine atoms. A hydrocarbon-based oil may contain ester, ether, amine and/or amide groups. The term silicone oil generally refers to an oil containing at least one silicon atom, and may contain one or more Si—O groups. The term fluoro oil generally refers to an oil containing at least one fluorine atom.

In some embodiments, a composition herein comprises a fatty alcohol. Fatty alcohols may have the structure R—OH where R is chosen from saturated and unsaturated, linear and branched radicals containing for example, 4 to 40 carbon atoms, 6 to 30 carbon atoms, or 12 to 20 carbon atoms. In at least one embodiment, R may be chosen from C12-C20 alkyl and C12-C20 alkenyl groups. R may or may not be substituted with at least one hydroxyl group. Non-limiting examples of fatty alcohols include lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

In some embodiments, a composition herein comprises a fatty acid. Fatty acids may include, for example, carboxylic acids, saturated or unsaturated, having for example, 6 to 30 carbon atoms, or 9 to 30 carbon atoms. Fatty acids may be chosen from myristic acid, palmitic acid, stearic acid, behenic acid, oleic acid, linoleic acid, linolenic acid and isostearic acid.

Volatile Oil

The term volatile oil generally refers to an oil (or non-aqueous medium) capable of evaporating on contact with the skin in less than one hour, at ambient temperature and atmospheric pressure. A volatile oil is a volatile cosmetic oil that is liquid at ambient temperature, having in particular a non-zero vapor pressure, at ambient temperature and atmospheric pressure, in particular having a vapor pressure ranging from 0.13 Pa to 40,000 Pa (10-3 to 300 mmHg), 1.3 Pa to 13,000 Pa (0.01 to 100 mmHg), or 1.3 Pa to 1300 Pa (0.01 to 10 mmHg). A volatile oil generally has a boiling point, measured at atmospheric pressure, ranging from about 150° C. to about 260° C., or ranging from about 170° C. to about 250° C.

A volatile oil may be a hydrocarbon-based or silicone oil. In some embodiments, a hydrocarbon-based volatile oil may be chosen from hydrocarbon-based oils having a flash point ranging from about 40° C. to about 102° C., about 40° C. to about 55° C., or about 40° C. to about 50° C.

Hydrocarbon-based volatile oils may contain 8 to 16 carbon atoms, which may be linear or branched, and mixtures thereof. Branched hydrocarbon-based volatile oils may include C8-C16 alkanes, such as C8-C16 isoalkanes (also referred to as isoparaffins), isododecane, isodecane, isohexadecane, an undecane/tridecane mixture, dodecane, tetradecane, and mixtures thereof; and C8-C16 branched esters, such as isohexyl neopentanoate, and mixtures thereof.

Non-Volatile Oil

A non-volatile oil may be chosen from carbon-based, hydrocarbon-based, silicone oils, and fluoro oils of mineral, animal, vegetable or synthetic origin, and mixtures thereof. For example, non-volatile hydrocarbon-based oils may include liquid paraffin or liquid petroleum jelly, isoeicosane, soya oil, perhydrosqualene, sweet almond oil, beauty-leaf oil, palm oil, grapeseed oil, sesame oil, maize oil, rapeseed oil, sunflower oil, cottonseed oil, apricot oil, castor oil, avocado oil, jojoba oil, olive oil or cereal germ oil; esters of lanolic acid, of oleic acid, of la uric acid, of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate, lactate, 2-diethylhexyl succinate, diisostearyl malate, glyceryl triisostearate, diglyceryl triisostearate; carbonates, such as dicaprylyl carbonate; ethers, such as dicaprylyl ether; higher fatty acids, such as myristic acid, palmitic acid, stearic acid, behenic acid, oleic acid, linoleic acid, linolenic acid, isostearic acid; higher fatty alcohols, such as cetanol, stearyl alcohol, oleyl alcohol, linoleyl, linolenyl alcohol, isostearyl alcohol, octyldodecanol.

In some embodiments, a composition herein is a transparent emulsion, which comprises at least one oil selected from liquid esters of saturated or unsaturated, linear or branched C1-C26 aliphatic monoacids or polyacids and of saturated or unsaturated, linear or branched C1-C26 aliphatic monoalcohols or polyalcohols, the total number of carbon atoms of the esters being greater than or equal to 10. In certain embodiments, for the esters of monoalcohols, at least one from among the alcohol and the acid from which the esters of the present invention are derived is branched. Monoesters of monoacids and of monoalcohols may include, for example, ethyl palmitate, ethyl hexyl palmitate, isopropyl palmitate, dicaprylyl carbonate, alkyl myristates such as isopropyl myristate and ethyl myristate, isocetyl stearate, 2-ethylhexyl isononanoate, isononyl isononanoate, isodecyl neopentanoate, and isostearyl neopentanoate.

Surfactants and/or Emulsifiers

A composition herein (e.g., a cosmetic composition, a sunscreen and/or sunblock composition, a topically-applied pharmaceutical composition) may comprise one or more surfactants and/or emulsifiers. For example, a composition herein may comprise one or more surfactants to obtain a transparent emulsion (e.g., a transparent oil-in-water emulsion). A surfactant may be present in a composition herein in an amount ranging from 0.01% by weight to about 10% by weight, from about 0.5% by weight to about 7% by weight, or from about 1% by weight to about 5% by weight, relative to the total weight of the composition. Surfactants may include nonionic surfactants, anionic surfactants, and mixtures thereof.

A surfactant may include, for example, a fatty acid ester of polyethylene glycol and/or a glyceryl ester as a nonionic surfactant. Examples of fatty acid ester of polyethylene glycol may include PEG-8 Stearate, PEG-6 Oleate, PEG-6 Isostearate, PEG-12 Isostearate, PEG-12 Diisostearate, PEG-8 Isostearate, PEG-8 Diisostearate, PEG-10 Isostearate, PEG-100 stearate, and the like. Examples of glyceryl ester may include glyceryl oleate, glyceryl monostearate (or glyceryl stearate), glyceryl monoisostearate, glyceryl monopalmitate, glyceryl monobehenate, and mixtures thereof.

In some embodiments, a composition herein comprises one or more nonionic surfactants. Non-limiting examples of nonionic surfactants include alkyl- and polyalkyl-esters of glycerol, polyglycerol ester of fatty acids, mixtures of alkyl- and polyalkyl-esters of glycerol with polyglyceryl, such as polyglyceryl-3 methylglucose distearate, oxyalkylenated (e.g., polyoxyethylenated), fatty acid esters of glycerol; oxyalkylenated fatty acid esters of sorbitan; oxyalkylenated (oxyethylenated and/or oxypropylenated) fatty acid esters (esters of polyethylene glycol and fatty acids); oxyalkylenated (oxyethylenated and/or oxypropylenated) fatty alcohol ethers; sugar esters, for instance sucrose stearate; fatty alcohol ethers of sugars, especially alkyl polyglucosides (APGs) such as decyl glucoside, lauryl glucoside, cetostearyl glucoside (e.g., as a mixture with cetostearyl alcohol), and arachidyl glucoside (e.g., in the form of a mixture of arachidyl alcohol), behenyl alcohol, arachidyl glucoside, lecithins and derivatives (e.g., biophilic), sugar esters, and sodium stearoyl lactylate.

In some embodiments, a composition herein comprises one or more anionic surfactants. Non-limiting examples of anionic surfactants include glyceryl stearate, PEG-100 stearate, Poloxamer 338, alkylamido ether sulfates, alkylaryl polyether sulfates, monoglyceride sulfates, sultanates, such as alkylsulfonates, alkylamide sultanates, alkylarylsulfonates, alpha-olefin sultanates, paraffin sultanates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates, N-acyl N-methyltaurates, N-acylisethionates, N-acyltaurates, salts of alkyl monoesters and polyglycoside-polycarboxylic acids, acyllactylates, mixed esters of organic acids with glycerol, such as glyceryl stearate citrate and as glyceryl stearate lactate, salts of D-galactoside uronic acids, salts of alkyl ether carboxylic acids, salts of alkyl aryl ether carboxylic acids, and salts of alkylamido ether carboxylic acids; or the non-salified forms of the above compounds, the alkyl and acyl groups the above compounds containing from 6 to 24 carbon atoms and the aryl group denoting a phenyl group. Some of the above compounds may be oxyethylenated and may comprise from 1 to 50 ethylene oxide units.

Anionic surfactants also may include anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl poly glucosides, and mixtures thereof.

Emulsifiers that can be used may include non-ionic or ionic emulsifiers (anionic, cationic or amphoteric). In various embodiments, non-ionic emulsifiers include polyalkylene glycol ethers of fatty alcohols comprising from 8 to 30 carbon atoms and preferably from 10 to 22 carbon atoms; alkyl esters of polyoxyalkylenated sorbitan and, in particular, polyoxyethylene, wherein the alkyl radical comprises from 8 to 30 carbon atoms and preferably from 10 to 22 carbon atoms; polyoxyalkylene alkyl esters and, in particular, polyoxyethylene, wherein the alkyl radical comprises from 8 to 30 carbon atoms and preferably from 10 to 22 carbon atoms; polyethylene glycols; polypropylene glycols; diethylene glycols; and mixtures thereof. In various embodiments, the emulsifier is polysorbate 20, ceteareth 20, diutan gum, carrageenan, gellan gum, welan gum, pectin, sclerotium gum, starch, or galactoarabinan. In various embodiments, the emulsifier is xanthan gum. The amount of emulsifier or emulsifiers is generally from 0.001% to 30% by weight, based on the total weight of the composition. In various embodiments, the emulsifier is present at greater than zero percent and less than about 2% by weight. In various embodiments, one or more emulsifiers are present at greater than zero percent and less than 1%, preferably about 0.2%. In various embodiments, cosmetic compositions as described herein have emulsion stability at 24 hours at least as stable as Anti-Aging Serum B (see Example 5).

Polymer

A composition herein (e.g., a cosmetic composition, a sunscreen and/or sunblock composition, a topically-applied pharmaceutical composition) may comprise one or more polymers. Suitable polymers include but are not limited to polylactic acid (PLA), poly C10-C30 alkyl acrylate, acrylates/C10-C30 alkyl acrylate crosspolymer, styrene/acrylates copolymer, lauryl methacrylate/glycol dimethacrylate crosspolymer, ammonium acryloyldimethyltaurate/vp copolymer, dimethicone/vinyl dimethicone crosspolymer, ammonium polyacryloyldimethyl taurate, aluminum starch octenylsuccinate and mixtures thereof.

Additive

A composition herein (e.g., a cosmetic composition, a sunscreen and/or sunblock composition, a topically-applied pharmaceutical composition, an anti-aging serum) may comprise one or more additives. For example, a composition may include one or more fragrances, vitamins (e.g., tocopherol, niacinamide, vitamin B3, vitamin B6), NAD-boosting compounds, (e.g. nicotinamide riboside (NR), nicotinamide mononucleotide (NMN), C6-C18 fatty acid nicotinate esters such as myristyl nicotinate or tetradecyl nicotinate), NAMPT inhibitors, preservatives (e.g., phenoxyethanol and salicylic acid), silicones (e.g., dimethicone, caprylyl methicone, vinyl dimethicone/methicone silsesquioxane crosspolymer), fatty compounds, fillers (e.g., silicas (e.g., silica silylate), mica, magnesium oxide, nylon-12, nylon-66, cellulose, talc, talc and methicone, talc and dimethicone, perlite, sodium silicate, pumice, PTFE, polymethyl methacrylate, alumina, calcium sodium borosilicate, magnesium carbonate), solvents (e.g., short-chain alcohols (e.g., ethanol), glycols, polyols, glycerin, caprylyl glycol, pentylene glycol, propylene glycol, butylene glycol), thickeners, organic or mineral additives, physical and/or chemical sunscreens, sequestering agents, antioxidants, insoluble active agents, liposoluble active agents, water-soluble active agents, moisturizers such as polyols (e.g., glycerol), pH adjusters (acids or bases), additional active agents (e.g., agents extracted from plants, agents resulting from biotechnology, disodium EDTA, triethanolamine, capryloyl salicylic acid, hydroxyethylpiperazine ethane sulfonic acid), mineral active agents, and/or tensioning agents.

Additives may be present at concentrations ranging from about 0.1% to about 90% by weight, from about 0.1% to 10% by weight, from about 1% to about 90% by weight, from about 5% to about 80% by weight, from about 10% to about 70% by weight, from about 15% to about 60% by weight, or from about 20% by weight to about 50% by weight, based on the total weight of the composition herein.

In certain implementations, a composition provided herein includes one or more sirtuin activators. A sirtuin activator sometimes is a phenol or a stilbenoid. A non-limiting example of sirtuin activator is resveratrol (3,5,4′-trihydroxy-trans-stilbene).

In certain implementations, a composition provided herein includes one or more anti-oxidants. An anti-oxidant sometimes is a free-radical scavenger, and sometimes is a phenol or a stilbenoid. Non-limiting examples of anti-oxidants include vitamin A, vitamin E, butylated hydroxytoluene (BHT), Urolithin A, and butylated hydroxyanisole (BHA). In various embodiments, the anti-oxidant improves stability or shelf-life of the composition. In various embodiments, the anti-oxidant provides protection against damage to cells or surfaces on which the composition is applied.

UV Filter

A composition herein (e.g., a cosmetic composition, a sunscreen and/or sunblock composition, a topically-applied pharmaceutical composition) may comprise one or more UV filter components. UV filters may be active in the UV-A and/or UV-B region. UV filters may be hydrophilic and/or lipophilic. UV filters may be solid or liquid.

Any suitable UV filter may be included in a composition herein. Suitable UV filters may include, for example, anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinal; cysteine; aromatic amino acids; peptides having an aromatic amino acid residue; and mixtures thereof.

In some embodiments, a UV filter may be chosen from butyl methoxydibenzoylmethane, ethylhexyl methoxycinnamate, homosalate, octocrylene, phenylbenzimidazole sulfonic acid, benzophenone-3, benzophenone-4, benzophenone-5, n-hexyl 2-(4-diethylamino-2-hydroxybenzoyl)benzoate, 1,r-(1,4-piperazinediyl)bis[1-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, l,l-dicarboxy(2,2′-dimethylpropyl)-4,4-diphenylbutadiene, 2,4-bis[5-1 (dimethylpropyl)benzoxazol-2-yl-(4-phenyl)imino]-6-(2-ethylhexyl)imino-1,3,5-triazine, camphor benzylkonium methosulfate, and mixtures thereof.

In some embodiments, compositions as described herein further comprise one or more compounds selected from a benzophenone or derivative thereof, a cinnamate compound or derivative thereof, a salicylate compound or derivative thereof, a mineral-based compound or derivative thereof, oxybenzone, avobenzone, octinoxate, octisalate, octocrylene, homosalate, titanium dioxide and zinc oxide. In various implementations, one or more or all of the following compounds are excluded: a benzophenone or derivative thereof, a cinnamate compound or derivative thereof, a salicylate compound or derivative thereof, a mineral-based compound or derivative thereof, oxybenzone, avobenzone, octinoxate, octisalate, octocrylene, homosalate, titanium dioxide and zinc oxide. In various implementations, one or more or all of the following compounds are either not present, or are present in an amount that does not provide for UV protection: a benzophenone or derivative thereof, a cinnamate compound or derivative thereof, a salicylate compound or derivative thereof, a mineral-based compound or derivative thereof, oxybenzone, avobenzone, octinoxate, octisalate, octocrylene, homosalate, titanium dioxide and zinc oxide.

Additional Implementations

In certain implementations, the cellular remains and/or secretions of bacteria in a composition provided herein is/are about 5% or less by weight in the composition. The cellular remains and/or secretions of bacteria sometimes is/are about 4% or less, about 3% or less, about 2% or less, about 1% or less, about 0.5% or less, about 0.25% or less, about 0.1% or less, about 0.05% or less, about 0.025% or less, about 0.01% or less, or about 0.005% or less, by weight, in the composition.

A composition provided herein often is stable for 24 hours or longer, and often has a shelf-life of 24 hours or longer. A composition provided herein sometimes includes xanthan gum in an amount greater than zero, and in an amount of about 0.2% by weight or less, and sometimes in an amount of about 0.1% or less, about 0.05% or less, about 0.025% or less, about 0.01% or less, about 0.005% or less, about 0.0025% or less, or about 0.001% or less, by weight, in the composition.

In certain implementations, a composition provided herein blocks blue spectrum ultraviolet light relative to a composition that does not include the cellular remains of the bacteria and/or secretions of the bacteria. Blue spectrum ultraviolet light generally is high energy light and can be in the wavelength range of about 380 nanometers to about 530 nanometers. A composition provided herein sometimes blocks, prevents transmission of and/or absorbs blue spectrum ultraviolet light. A composition provided herein sometimes blocks about 0.1% to about 10% of blue spectrum ultraviolet light, sometimes blocks about 0.5% to about 5% blue spectrum ultraviolet light, sometimes blocks about 1% to about 3% blue spectrum ultraviolet light and sometimes blocks about 2% blue spectrum ultraviolet light. An amount of light blocked sometimes is an amount of solar spectral irradiance blocked by a composition provided herein.

### Methods of Manufacture

In various embodiments, provided are methods of manufacturing a composition by obtaining bacteria, e.g., of the same strain of Bacillus pumilus deposited under ATCC accession number PTA-126909, optionally rendering the bacteria non-viable (e.g., through lyophilization, lysis, sonication, microfluidization, or the like), and adding the bacteria or its non-viable components to a carrier to yield the composition. The carrier may include any components that are commonly used in the formulation of final compositions, depending on the intended use of the composition. Processing of the bacteria from growth media may include processing steps such as lyophilization, lysis, centrifugation (i.e., spinning-down), decantation, physical shearing, or the like. In various embodiments, microfluidization is included to improve UV absorbance, homogeneity, and/or reproducibility.

In various embodiments, provided herein are methods of directed evolution to produce a bacteria with resistance to ultraviolet light, comprising obtaining a sample of Bacillus pumilus, growing the sample in ultraviolet light at greater than or equal to 700 joules/m2 of exposure (fluence), optionally ultraviolet light at greater than or equal to 900 joules/m2 of exposure (fluence), and isolating viable bacteria surviving such growth conditions, wherein the isolated viable bacteria exhibit greater UV absorption compared to untreated Bacillus pumilus. In various embodiments, methods are provided for (1) isolating surviving microorganisms and culturing the microorganisms under UVC radiation shielding and exposure to an artificial source of ultraviolet light between 700 joules/m2 (fluence) and about 900 joules/m2 (fluence); (2) isolating surviving microorganisms of (1) and culturing the microorganisms under UVC radiation shielding and exposure to an artificial source of ultraviolet light between 700 joules/m2 (fluence) and about 900 joules/m2 (fluence), wherein the amount of UVC radiation shielding is less than the amount of UVC radiation shielding in (1); and optionally repeating (2) one or more times using the microorganisms surviving after each iteration for culturing in the next iteration, wherein at each culturing the amount of UVC radiation shielding is less than the amount of the previous iteration. In various embodiments, the number of iterations is at least 5, at least 10, or at least 15. Such evolved bacteria may have one or more phenotypic differences from the source organism, or one or more genotypic differences from the source organism, or both. In certain implementations, the bacteria are phenotypically distinct by visual inspection from progenitor bacteria. In certain implementations, stock samples of bacteria are frozen for storage, thawed from storage, screened for viability including exposure to an artificial source of ultraviolet light between 700 joules/m2 (fluence) and about 900 joules/m2 (fluence) for a time sufficient to verify growth in the presence of ultraviolet light, inoculated into growth media in a reactor vessel for a time sufficient for the growth cycle to be complete based on the amount of growth media, concentrated to remove growth media, for example by one or more rounds of centrifugation, washed with water, and lysed to remove viability, for example through microfluidization, and reconstituted with water to achieve a uniform percentage of solids to liquids by weight, for example between 0.01% by weight to about 10% by weight solids to liquids, preferably about 0.5% by weight, or about 1% by weight, or about 2% by weight, or about 3% by weight, or about 4% by weight, or about 5% by weight. In various embodiments, samples are screened for any contaminating bacteria or pathogens, and contaminated samples are discarded. In various implementations, an antifoaming agent is added to assist in the processing steps as described herein. In various implementations, post-lysis samples are screened for non-viability following lysis.

### Methods of Use

A composition described herein can be incorporated into an article or can be applied to an article, for example, where the article sometimes is a substrate. In various embodiments, provided herein are methods of providing protection from ultraviolet light comprising administering (e.g., applying) a composition as described herein to the surface of a substrate. In various embodiments, provided herein are methods of altering effects and/or transmission of ultraviolet light, comprising embedding or integrating a composition as described herein into a substrate. The substrate may be in non-limiting examples (i) a biological substrate, such as human skin, or (ii) a non-living substrate, such as a rigid article (e.g., a wall on a building or window where the composition is applied as a paint), or a flexible article (e.g., a textile, fabric, fur, hair, garment, shade). The composition sometimes is applied as a coating layer on the surface of the article and/or integrated into the article. In certain implementations, the composition is applied as a paint, stain, lacquer, varnish, glaze, ink, a UV-stabilizer, a textile-treatment composition, a leather-treatment composition, a hair-treatment composition, and/or a fur-treatment composition. In certain implementations, the composition is added to dietary supplements, post-biotic by-products, and/or fertilizer or plant-growth compositions.

In certain implementations, a composition described herein is utilized to increase an amount of skin hyaluronic acid. Provided in certain aspects is a method for increasing an amount of skin hyaluronic acid, which includes administering topically a composition described herein to skin of a subject in an amount effective to increase the amount of skin hyaluronic acid. A composition described herein can be administered topically to skin of a subject in need thereof, and an amount of hyaluronic acid can increase on and/or in the skin of the subject.

In certain implementations, a composition described herein is utilized to decrease an amount of elastin. Provided in certain aspects is a method for decreasing an amount of skin elastin, which includes administering topically a composition described herein to skin of a subject in an amount effective to decrease the amount of skin elastin. A composition described herein can be administered topically to skin of a subject in need thereof, and an amount of elastin can decrease on and/or in the skin of the subject.

In certain implementations, a method for boosting sun protection factor (SPF) in a composition is provided, that includes combining a composition described herein with a base composition chosen from a cosmetic composition, a sunscreen and/or sunblock composition, and a topically-applied pharmaceutical composition, thereby providing a combined composition, where the base composition has a first SPF value prior to the combining, and the combined composition has a second SPF value greater than the first SPF value. As a SPF booster, a composition provided herein may increase SPF efficiency by about 50% to about 200% depending on the base composition. This increase may reduce the amount of SPF active ingredient in a base composition needed from about 15%-20% to about 8%-12% for day wear products and to about 6%-10% for beach products. As a SPF booster, bacterial lysate components (e.g., cellular remains and secretions of bacteria, including, for example Bacillus Lysate) can constitute about 0.01% to about 5% by weight, or about 1% to about 3% by weight, or about 0.1%, about 0.5%, about 0.8%, about 1%, about 2%, or about 3% by weight, of a combined composition. As a SPF booster, a composition described herein may increase UV absorption, may increase film thickness on skin, may increase UV scattering, may increase UV coverage by extending the critical wavelength, may improve UV stability, and/or may reduce UV induced skin erythema, relative to the base composition. In various embodiments, compositions as described herein, including for example Bacillus Lysate, boost the SPF value of an existing SPF composition with an SPF from 15-20 up to SPF 21-40, or any value in between, such as SPF 22, 23, 24, 25, 26, 27, 28, 29, 30, 31, 32, 33, 34, 35, 36, 37, 38, or 39. In various embodiments, a composition described here when used as an additive to a composition with a baseline SPF from 15-20 will contain from 1%-10%, optionally about 2%, or about 5%, or about 7%, by weight of solids to liquids prior to addition to the baseline SPF composition.

In certain implementations, provided is a method for protection from ultraviolet light, comprising administering a composition described herein to skin of a subject. The composition can be administered any suitable number of times per day (e.g., once per day, twice per day, three or more times per day multiple times per day). A subject can be a human or non-human subject. A non-human subject sometimes is a mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an infant, juvenile, child, adult). A composition can be administered by any suitable method, non-limiting examples of which include administration by hand, spray, applicator (e.g., a roller, brush, pad) and the like.

In certain implementations, provided is a method for protecting skin from HEV light, comprising administering a composition described herein to a surface and/or skin of a subject.

In certain implementations, provided is a method for increasing activity of sirtuin enzymes in skin, comprising administering a composition described herein to a surface and/or skin of a subject. In certain implementations, provided is a method for increasing activity of sirtuin enzymes in skin, comprising administering a composition described herein to a surface and/or skin of a subject, wherein the composition is essentially free of sirtuin activators other than the cellular remains and debris as described herein, for example Bacillus Lysate. In certain implementations, provided is a method for increasing activity of sirtuin enzymes in skin, comprising administering a composition comprising both i) the cellular remains and debris as described herein, for example Bacillus Lysate, and ii) one or more sirtuin-enzyme activating components, such as resveratrol.

In certain implementations, provided is a method for reducing advanced glycation end products (AGEs) in skin, comprising administering a composition described herein to a surface and/or skin of a subject.

In certain implementations, provided is a method for providing anti-aging effects to human skin, comprising administering a composition described herein to skin of a human subject. In certain implementations, provided is a method of both increasing activity of sirtuin enzymes and providing anti-aging effects in human skin, comprising administering a composition described herein to skin of a human subject.

In certain implementations, provided is a method for providing anti-aging effects in skin, comprising administering a composition comprising both i) the cellular remains and debris as described herein, for example Bacillus Lysate, and ii) one or more NAD-boosting components, such as nicotinamide riboside (NR) or nicotinamide mononucleotide (NMN) or myristyl nicotinate.

### INCORPORATION BY REFERENCE

All US patents and US and PCT published patent applications and non-patent literature mentioned in this specification are herein incorporated by reference to the same extent as if each independent patent and publication was specifically and individually indicated to be incorporated by reference.

### EXAMPLES

### Example 1: Directed Evolution of Bacterial Strains

First iteration: Strains of B. pumilus are spread on Tryptic Soy Agar petri plates and immediately placed under a Biosafety Cabinet UVC lamp (254 nm) for varying amounts of time, up to an estimated 900J/m² of fluence. Colonies that result from surviving cells are used to inoculate test tubes of 5 mL Tryptic Soy Broth (TSB), which are grown overnight at 37° C. with shaking. For storage, 1 mL of the dense culture is added to 1 mL of 50% glycerol/50% water (v:v) and frozen at −80° C. Initial strains may include B. pumilus obtained from environmental sources, or B. pumilus obtained from ATCC collections, such as ATCC deposit PTA-7603, or strains derived from private or commercial collections, or as described in the literature, for example SAFR-032, UV-Space (56T-2), UV-Mars (183T-1), Dark-Space (40T-5), and Dark-Mars (168T-5).

Subsequent iterations: Frozen stocks are used to inoculate TSB overnight, which are then diluted 1:1000 in 10-fold diluted TSB (10% TSB). Dilute media is used to reduce interference from full strength media with high UVC absorbance from an abundance of aromatic amino acids such as tryptophan. 2.5 mL of inoculated 10% TSB is distributed into 24-well plates in patterns that control for slight differences in UVC incidence. Pre-sterilized plates containing tiny bleach-sterilized magnetic stir bars are covered with various amounts of UVC-translucent shielding (for example, 12 layers of clear plastic bags in which disposable petri dishes are “sleeved”). Colony-forming units are determined at initiation (time 0 hr), and subsequent time points (usually time 6 hr and time 23 hr) via tittering (serial dilution and plating). Upon conclusion of an iteration, the surviving B. pumilus that has grown visible on the TSA plates may be heat-sterilized with an inoculating loop, and a swath may be collected from the resulting genetically-diverse lawn, and inoculated in 100% TSB overnight. Plating, then growing in rich media, allows for two physiological “resets”, so that the cells that survive the succeeding round of UVC radiation may do so due to a genetic rather than physiological adaptation. This process may be used at a subsequent time point to inoculate a new sterile 24-well plate, half of which has the same amount of shielding (e.g., 12 layers of petri plate bag plastic) or a reduced amount (e.g., 11 layers of petri plate plastic). Wherever possible, the wells most directly under the light that show growth are used to inoculate the next iteration.

After less than one layer of petri plate bag shielding is required while still achieving sufficient growth, thinner shielding (e.g., cling wrap) is used, with ethanol sterilization to prevent cross-contamination. UVC is increased by placing the magnetic stirrer on supports. When the amount of UVC is checked, the luminometer is covered with the equivalent amount of shielding and placed at the equivalent distance from the Biosafety Cabinet UVC bulb.

Between iterations, B. pumilus grows rapidly so that titers show visible “lawns” within a few hours of tittering. These “lawns” are used to inoculate 5 mL 100% TSB test tubes, which are grown at 37° C. with shaking for an additional couple of hours. Thus, initial inocula are titered at time 0 hr at the end of the day, and time ˜18 hr the next morning.

After a specific number of iterations such as Iteration 8 or Iteration 30, cells form visually apparent clumps, especially (but not exclusively) when grown under UVC. With each successive iteration, titers of dense B. pumilus-evolved strains show lower and lower cfu/mL; indicating the possibility that each colony forms from various numbers such as ten to one hundred bacterial cells instead of one bacterial cell. In various iterations, colonies may be screened for the presence of B. cereus as a contaminant, and such colonies may be removed or not selected for subsequent iterations.

Upon reaching a desired level of UV absorbance or resistance, bacterial cells may be frozen for storage. Such stored samples may be thawed and propagated to achieve useful quantities of cellular material for formation of compositions, such as Bacillus Lysate, obtained as described herein. Prior to propagation, stored samples may be screened for viability by exposing the samples in growth media to UV light to verify UV absorbance or resistance is maintained upon removal from storage, with colonies that survive UV screening greater than or equal to 700 joules/m² of exposure (fluence), optionally approximately 900 joules/m² of exposure (fluence), serving as the source material for propagation or amplification in growth reactors of various sizes, for example 50 L, 500 L and 5000 L.

### Example 2: UV Absorbance Testing

General procedure for preparing samples for UV absorbance testing: cells are grown in 10% tryptic soy broth (TSB) in test tubes for 1, 2, and 3 days. Samples are taken from each day for each strain and either left as-is, heat-killed by boiling at 100° C. for 10 minutes, sonicated for 5 minutes total in 30-second on/off cycles at an amplitude of 1, heat-killed and sonicated before flash-freezing and lyophilizing, or microfluidized, and stored as a solid (amorphous powder).

### Measuring the Absorbance of Cells

Amorphous powder (10.0 mg) was weighed into a 2 mL Eppendorf tube and re-suspended in 1 mL deionized water (diH₂O) resulting in a 10.0 mg/mL solution, with vortexing for complete dispersal/solubilization. The sample was serially diluted to 1.0 mg/mL by pipetting 100 microliters into 900 microliters diH₂O in a second 2 mL Eppendorf tube. Additional serial dilutions (0.5 mg/mL; 0.1 mg/mL) were prepared by pipetting 250 microliters of the 1.0 mg/mL solution into 250 microliters diH₂O in a third 2 mL Eppendorf tube and by pipetting 100 microliters of the 1.0 mg/mL solution into 900 microliters diH₂O in another Eppendorf tube, respectively.

A comparative sample of oxybenzone was prepared by weighing out 10.0 mg oxybenzone into a 2 mL Eppendorf tube and re-suspending in 1 mL 200 proof ethanol resulting in a 10.0 mg/mL solution. The sample was serially diluted to 1.0 mg/mL by pipetting 100 microliters of the 10.0 mg/mL solution into 900 microliters 200 proof ethanol in a second 2 mL Eppendorf tube. Additional serial dilutions (0.1 mg/mL; 0.01 mg/mL) were prepared by pipetting 100 microliters of the 1.0 mg/mL solution into 900 microliters of 200 proof ethanol in a third 2 mL Eppendorf tube, and by pipetting 100 microliters of the 0.1 mg/mL solution into 900 microliters of 200 proof ethanol in a subsequent Eppendorf tube, respectively.

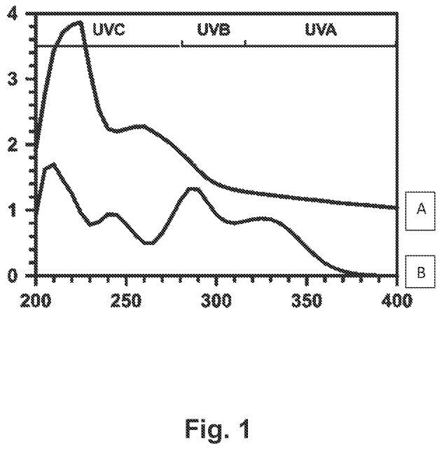

Using a UV transparent flat bottom 96 well plate, 100 microliter samples were pipetted into 3 wells each (e.g., test sample, comparative oxybenzone sample, diH₂O blanks, and 200 proof ethanol blanks). The absorption spectrum of each well was measured using a Molecular Devices SpectraMax Plus plate reader. The absorption of each sample was measured from 200 nm to 400 nm in 5 nm increments. The raw spectra data of each sample was imported into an Excel spreadsheet, where the absorbances of the wells containing only diH₂O and only 200 proof ethanol (the respective blanks) were subtracted as background from the absorbances of the wells containing dilutions of test material. The absorbances of the wells were then divided by their concentrations (e.g., a sample at 0.25% was divided by 0.25) and the averages and standard deviations were calculated for all wells pertaining to a sample. The average absorption at each measured wavelength was plotted for each sample. See FIG. 1 showing the UV spectra for a test composition (Trace A) versus a comparative sample (oxybenzone), where the Y-axis is UV absorbance (scale of 0-4) and the X-axis is wavelength (200-400 nm). Trace A is a test composition at 1.0 mg/mL; and Trace B is oxybenzone at 0.1 mg/mL.

### Example 2A: Alternative Lysis Procedure

Samples are prepared by microfluidization of bacterial cells according to the following procedure: A 20 g pellet (˜5.5E+10 cells) is diluted in diH₂O to a total volume of 250 mL, creating a cell suspension of approximately 2.2E+08 cells/mL. 100 mL of this dilution is used with a laboratory-scale microfluidizer, and 150 mL is used with a pilot scale microfluidizer. Samples are obtained after 10 rounds of microfluidization from each microfluidizer, and samples are examined for homogeneity by visual inspection under magnification.

### Example 3: Scale Up Optimization

A strain, obtained as described in Example 1, is selected for scale-up and production optimization. Flask type, media volume and strength, incubation time, and incubation temperature (e.g., at 37° C.) are compared for the optimal growth.

Optimization for laboratory flask type: 2,800 mL Fernbach flasks containing 250 mL, 500 mL or 1,000 mL of 10% TSB are compared to 2,500 mL Low Form flasks containing 500 mL, 1,000 mL or 1,500 mL of 10% TSB. Five 50 mL samples are taken from each flask after 1, 2 and 3 days of growth with shaking at 37° C., and compared for bacterial density.

Optimization for post-growth treatment: five post-growth treatment types are tested: (i) centrifugation at 5,000×g for 10 minutes, (ii) centrifugation, washing with sterile diH₂O, then centrifuging again, (iii) centrifugation, washing, centrifugation, then resuspension of the pellet in ˜10 mL diH₂O followed by sonication for 5 minutes in 30-second on/off cycles at an amplitude of 1, (iv) centrifugation, washing, centrifugation, sonication, then autoclaving and (v) centrifugation, then resuspension of the pellet in ˜10 mL diH₂O followed by autoclaving. All samples are flash-frozen in liquid nitrogen after receiving their respective post-growth treatments and stored at −80° C. pending lyophilization, microfluidization, or the like, and measurement of UV absorbance spectra, measured by the same method described in Example 2. A process described in this Example 3 can be utilized to manufacture a Bacillus Lysate.

### QA/QC for Batches

Batches are grown and treated according to the following procedure: The cells are grown in 1,500 mL of 10% TSB in 2,500 mL Pyrex Low Form Culture Flasks for one day. The batches are autoclaved, centrifuged, lyophilized, and re-suspended at 15% weight-to-volume (w:v) in diH₂O, sonicated, and lyophilized again before storing at −80° C. A sample of the sonicated material may be re-suspended in diH₂O at 0.25% w:v to compare to the performance of previous batches. Batches that perform according to specifications may be retained in cold storage (−80° C.).

Where visual inspection of batches or variation of results within a batch is found, the batch may be physically ground with a grinder until physical homogeneity for the batch is achieved. Alternatively, a batch may be subjected to microfluidization.

### Example 4: Formulation

### Suspending Cells in Foundation or Lotion for SPF Testing

Powdered bacterial components that pass the QA/QC cutoff for performance are re-suspended in foundation or lotion at concentrations of 15% or 25% w:v and either sonicated or left as-is. Commercially available lotion includes AVEENO® Baby Daily Moisture Lotion. Commercially available foundation includes FLOWER Beauty® Light Illusion with Broad Spectrum SPF 18. Samples are tested for SPF determination, for example a sonicated 15% resuspension in foundation.

### Example 5: Anti-Aging Serums

Provided hereafter is a description of components present in phases that are combined to prepare Anti-Aging Serum A.

| | Wt. % |
|---|---|
| PHASE A | |
| Water | To make |
| | 100% |
| Phenoxyethanol (and) ethhylhexl glycerin (Euxyl) | 1.000 |
| Hydroxyethyl Cellulose (Natrosol 250 HHR) | 0.500 |
| Glycerin | 3.000 |
| Propanediol (Zemea) | 1.500 |
| Sodum Hyaluronate | 1.500 |
| PHASE B | |
| Triticum Vulgare (Wheat) Germ Oil | 1.500 |
| Caprylic Capric Triglyceride (CCT) | 3.000 |
| Ferulic acid (iWhite FA) | 0.500 |
| Hydrolyzed Jojoba Esters (and) Water (Floraesters | 1.000 |
| K-20W) | |
| PHASE C | |
| Water | 10.000 |
| Mannitol (and) Yeast Extract (VITACELL ™ LS | 1.000 |
| 7979) | |
| Nannochloropsis Oculata Extract (and) Pullulan | |
| (and)Aqua | |
| (and)Phenoxyethanol (and) Sodium Benzoate | |
| (and) | |
| Glycerin (and) Aqua (and) Ulmus Fulva Bark | 2.000 |
| Extract | |
| Bacillus Lysate (BL) liquid | 2.000 |
| PHASE D | |
| Ethanol | 5.000 |
| Resveratrol | 0.500 |
| Urolithin A | 0.500 |
| PHASE E | |
| Pelargonium graveolens flower oil | 0.150 |

### Procedure:

1—In a vessel, weigh water, Euxyl and Natrosol, mix well and heat to ˜50-55° C.
2—Once Natrosol is fully hydrated and a gel is formed, remove from heat and let cool to room temperature and add remaining phase A components.
3—Premix Ferulic acid with Wheat germ oil and CCT to dissolve, then add jojoba ester. Add the mixture to phase A while mixing.
4—Premix C, add to main batch while mixing.
5—Premix D, add to main batch. Then add E. Mix until uniform.

Provided hereafter is a description of components present in phases that are combined to form Anti-Aging Serum B.

| | Wt. % |
|---|---|
| PHASE A | |
| Water | 58.410 |
| Phenoxyethanol (and) ethhylhexl glycerin | 1.000 |
| Hydroxyethyl Cellulose (Natrosol 250 HHR) | 0.200 |
| Xanthan gum | 0.200 |
| Glycerin | 2.500 |
| Propanediol (Zemea) | 1.000 |
| Sodum Hyaluronate | 1.500 |
| PHASE B | |
| Cetearyl Olivate (and) Sorbitan Olivate (Olivem | 3.000 |
| 100) | |
| Glyceryl Stearate | 0.500 |
| Stearic acid | 0.500 |
| Triticum Vulgare (Wheat) Germ Oil | 2.000 |
| Caprylic Capric Triglyceride | 2.500 |
| Ferulic acid (iWhite FA ) | 0.500 |
| PHASE C | |
| Water | 5.000 |
| Mannitol (and) Yeast Extract (VITACELL ™ LS | 1.000 |
| 7979) | |
| Nannochloropsis Oculata Extract (and) | 0.500 |
| Pullulan (and)Aqua | |
| (and)Phenoxyethanol (and) Sodium Benzoate | |
| (and) | |
| Glycerin (and) Aqua (and) Ulmus Fulva Bark | 1.000 |
| Extract | |
| Bacillus Lysate (BL) liquid | 2.000 |
| PHASE D | |
| Ethanol | 5.000 |
| Resveratrol | 0.500 |
| Urolithin A | 0.300 |
| PHASE E | |
| Water | 10.000 |
| Niacin (vitamin B3) | 0.090 |
| Pyridoxine HCl (vitamin B6) | 0.300 |
| PHASE F | 0.500 |
| Pelargonium graveolens flower oil | |

### Procedure:

1—In a vessel, weigh phase A, heat to ˜70-75° C. while mixing vigorously
2—In another vessel, weigh phase B, heat to ˜70-75° C., add to A, homogenize.
3—Start cooling the batch.
4—Premix water and Vitacell, add to main batch while mixing, add remaining phase C ingredients. Add mixture to main batch, mix well until uniform.
5—Premix D, add to main batch while mixing.
6—Heat water of phase E to ˜80-85°, then add niacin. Cool mixture to ˜50° C., add pyridoxine, mix well, add to main batch, mix well until uniform. Then add F.

Provided hereafter is a description of components present in phases that are combined to form Anti-Aging Serum C:

| | Wt % |
|---|---|
| PHASE A | |
| Water | 63.950 |
| Phenoxyethanol (and) ethhylhexl glycerin | 1.000 |
| Hydroxyethyl Cellulose (Natrosol 250 HHR) | 0.200 |
| Xanthan gum | 0.200 |
| Glycerin | 3.000 |
| Propanediol (Zemea) | 1.500 |
| Sodum Hyaluronate (ActiqueHyal 1%) | 1.500 |
| Hydrolyzed Jojoba Esters (and) Water (Floraesters K- | 1.000 |
| 20W) | |
| PHASE B | |
| Triticum Vulgare (Wheat) Germ Oil | 1.500 |
| Caprylic Capric Triglyceride | 3.000 |
| Ferulic acid (iWhite FA ) | 0.500 |
| Urolithin A | 0.500 |
| PHASE C | |
| Polyglyceryl-6 Caprylate (and) Polyglyceryl-3 Cocoate | 1.500 |
| (and) Polyglyceryl-4 Caprate (and) Polyglyceryl-6 | |
| Ricinoleate (TEGO ® Solve 61) | |
| PHASE D | |
| Water | 10.000 |
| Mannitol (and) Yeast Extract (VITACELL ™ LS 7979) | 1.000 |
| Nannochloropsis Oculata Extract (and) Pullulan | 1.000 |
| (and)Aqua | |
| (and)Phenoxyethanol (and) Sodium Benzoate (and) | |
| Glycerin (and) Aqua (and) Ulmus Fulva Bark Extract | 1.000 |
| Bacillus Lysate (BL liquid) | 2.000 |
| PHASE E | |
| Ethanol | 5.000 |
| Resveratrol | 0.500 |
| PHASE F | |
| Pelargonium graveolens flower oil | 0.150 |
| | 100.000 |

The formulation procedure for Anti-Aging Serum C is similar to the formulation procedures described above for Anti-Aging Serum A and for Anti-Aging Serum B.

Provided hereafter is a description of components present in phases that are combined to form Anti-Aging Serum D:

PHASE A

| | |
|---|---|
| Water | 58.410 |
| Phenoxyethanol (and) ethhylhexl glycerin | 1.000 |
| Hydroxyethyl Cellulose (Natrosol 250 HHR) | 0.200 |
| Xanthan gum | 0.200 |
| Glycerin | 2.500 |
| Propanediol (Zemea) | 1.000 |
| Sodum Hyaluronate | 1.500 |

PHASE B

| | |
|---|---|
| Cetearyl Olivate (and) Sorbitan Olivate (Olivem 100) | 3.000 |
| Glyceryl Stearate | 0.500 |
| Stearic acid | 0.500 |
| Triticum Vulgare (Wheat) Germ Oil | 2.000 |
| Caprylic Capric Triglyceride | 2.500 |
| Ferulic acid (iWhite FA ) | 0.500 |

PHASE C

| | |
|---|---|
| Water | 5.000 |
| Mannitol (and) Yeast Extract (VITACELL ™ LS 7979) | 1.000 |
| Nannochloropsis Oculata Extract (and) Pullulan | 0.500 |
| (and)Aqua (and)Phenoxyethanol (and) Sodium Benzoate | |
| Potassium Sorbate and Glycerin (and) Aqua (and) Ulmus | 1.000 |
| Fulva Bark Extract | |
| BL liquid (Bacillus Lysate) | 2.000 |

PHASE D

| | |
|---|---|
| Ethanol | 5.000 |
| Resveratrol | 0.500 |
| Urolithin A | 0.300 |

PHASE E

| | |
|---|---|
| Water | 10.000 |
| Niacin | 0.090 |
| Pyridoxine HCl | 0.300 |

PHASE F

| | |
|---|---|
| Pelargonium graveolens flower oil | 0.500 |

The formulation procedure for Anti-Aging Serum D is similar to the formulation procedures described above for Anti-Aging Serum A and for Anti-Aging Serum B.

### Comparative Stability of Emulsion (at 24 Hours):

Anti-aging Serum A in the absence of xanthan gum: +
Anti-aging Serum B with the presence of xanthan gum: ++
Anti-aging Serum C: ++
Anti-aging Serum D: ++

### Example 6: In-Vitro High Energy Visible Light (HEV 380-530 nm Blue Light) Screening

In this Example, a Bacillus Lysate described herein was evaluated according to the in vitro screening procedures described below.

### Screening Methods

The Bacillus Lysate sample was evaluated using a modified (single substrate, 1 scan, instead of three substrates, 15 scans) High Energy Visible Light Protection Factor (HEVPF) method (Advanced Science Laboratories, Inc.) to evaluate the sample's ability to protect against High Energy Visible light (380-530 nm) radiation.

### Results

Test Conditions:
Substrate: 1 HD 6u PMMA Plate
Application Rate: 1.3 mg/cm²
Dry Time: 15-30 min
Pre-irradiation Dose: 4 MEDs=800J/m²-eff

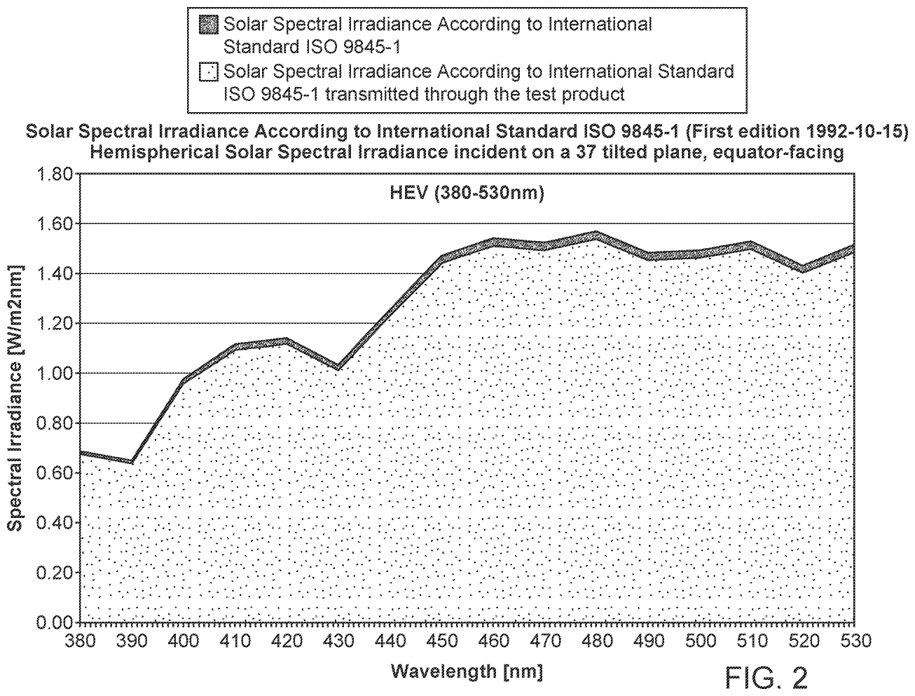

Test Results:
In-House In-Vitro Protection Factor (HEV-PF): 1.02
Percent Solar Spectral Irradiance Blocked (% SSI-HEV): 2.00% (see FIG. 2)

### Technical Summary

### Certificates of Spectral Measurement

Model 16S-300-003 V4.0

| Total Irradiance (250-1400 nm) | 2.47E−02 | W/cm² |
|---|---|---|
| Total UV Irradiance (250-400 nm) | 2.38E−02 | W/cm² |
| UVC Irradiance (250-290 nm) | 2.89E−08 | W/cm² |
| UVB Irradiance (290-320 nm) | 2.30E−03 | W/cm² |
| UVA Irradiance (320-400 nm) | 2.15E−02 | W/cm² |
| UVA2 Irradiance (320-340 nm) | 6.12E−03 | W/cm² |
| UVA1 Irradiance (340-400 nm) | 1.54E−02 | W/cm² |
| Visible + NIR Irradiance (400-1400 nm) | 8.84E−04 | W/cm² |
| % UVC | 0.0001% | |
| % UVB | 9.3% | |
| % UVA | 87.1% | |
| % Visible + NIR (400-1400 nm) | 3.6% | <5% |
| SED (seconds) | 64.0 | Seconds |
| Erythemal Effective Irradiance | 1.56E−04 | W/cm² |
| % Erythemal Contribution (FDA 2019) | | |
| % RCEE <290 nm (<0.1%) | 0.02% | Passed |
| 290 nm-300 nm (1.0%-8.0%) | 4.4% | Passed |
| 290 nm-310 nm (49.0%-65.0%) | 55.4% | Passed |
| 290 nm-320 nm (85.0%-90.0%) | 86.3% | Passed |
| 290 nm-330 nm (91.5%-95.5%) | 92.4% | Passed |
| 290 nm-340 nm (94.0%-97.0%) | 94.9% | Passed |
| 290 nm-400 nm (99.9%-100.0%) | 99.98% | Passed |
| % UVA2/Total UV (≥20%) | 25.7% | Passed |
| (320-340 nm/290-400 nm) | | |
| % UVA1/Total UV (≥60%) | 64.6% | Passed |
| (340-400 nm/290-400 nm) | | |
| Total Irradiance Limit (250-1400 nm) | | |
| FDA 2011 (<1500 W/m²) | 246.82 | Passed |

LS-1000-6S-UV

| Position | Center | U12 | U03 | U06 | U09 | |
|---|---|---|---|---|---|---|
| Total Irradiance (250-1400 nm) | 7.88E−03 | * | * | * | * | W/cm² |
| Total UV Irradiance (250-400 nm) | 7.49E−03 | 7.50E−03 | 7.31E−03 | 7.59E−03 | 7.67E−03 | W/cm² |
| UVC Irradiance (250-290 nm) | 1.32E−08 | 1.51E−08 | 2.59E−08 | 1.40E−08 | 1.65E−08 | W/cm² |
| UVB Irradiance (290-320 nm) | 7.00E−04 | 6.94E−04 | 6.76E−04 | 6.93E−04 | 7.06E−04 | W/cm² |
| UVA Irradiance (320-400 nm) | 6.79E−03 | 6.81E−03 | 6.63E−03 | 6.90E−03 | 6.97E−03 | W/cm² |
| UVA2 Irradiance (320-340 nm) | 1.77E−03 | 1.78E−03 | 1.73E−03 | 1.79E−03 | 1.81E−03 | W/cm² |
| UVA1 Irradiance (340-400 nm) | 5.02E−03 | 5.03E−03 | 4.90E−03 | 5.11E−03 | 5.16E−03 | W/cm² |
| Visible + NIR Irradiance | 3.89E−04 | * | * | * | * | W/cm² |
| (400-1400 nm) | | | | | | |
| % UVC | 0.0002% | * | * | * | * | |
| % UVB | 8.9% | * | * | * | * | |
| % UVA | 86.2% | * | * | * | * | |
| % Visible + NIR (400-1400 nm) | 4.96% | * | * | * | * | |
| SED (seconds) | 215.0 | 219.4 | 227.6 | 223.8 | 217.96 | seconds |
| Erythemal Effective Irradiance | 4.65E−05 | 4.56E−05 | 4.39E−05 | 4.47E−05 | 4.59E−05 | W/cm² |
| % Erythemal Contribution (FDA 2019) | | | | | | |
| % RCEE <290 nm (<0.1%) | 0.03% | 0.03% | 0.06% | 0.03% | 0.04% | Passed |
| 290 nm-300 nm (1.0%-8.0%) | 4.1% | 3.9% | 3.7% | 3.5% | 3.7% | Passed |
| 290 nm-310 nm (49.0%-65.0%) | 54.2% | 53.4% | 52.9% | 52.3% | 52.9% | Passed |
| 290 nm-320 nm (85.0%-90.0%) | 86.2% | 85.9% | 85.7% | 85.4% | 85.7% | Passed |
| 290 nm-330 nm (91.5%-95.5%) | 92.3% | 92.2% | 92.0% | 91.9% | 92.0% | Passed |
| 290 nm-340 nm (94.0%-97.0%) | 94.7% | 94.6% | 94.5% | 94.4% | 94.5% | Passed |
| 290 nm-400 nm (99.9%-100.0%) | 99.97% | 99.97% | 99.94% | 99.97% | 99.96% | Passed |
| % UVA2/Total UV (≥20%) | 23.6% | 23.7% | 23.7% | 23.6% | 23.6% | Passed |
| (320-340 nm/290-400 nm) | | | | | | |
| % UVA1/Total UV (≥60%) | 67.1% | 67.0% | 67.1% | 67.3% | 67.2% | Passed |
| (340-400 nm/290-400 nm) | | | | | | |
| Total Irradiance Limit (250-1400 nm) | | | | | | |
| FDA 2011 (<1500 W/m²) | 78.8 | * | * | * | * | Passed |

ATLAS Suntest CPS+

| Position | S1 | S2 | S3 | S4 | M1 | M2 | |
|---|---|---|---|---|---|---|---|
| Total Irradiance (250-800 nm) | 6.65E−02 | 6.90E−02 | 6.67E−02 | 6.19E−02 | 7.11E−02 | 7.11E−02 | W/cm² |
| UVC Irradiance (250-290 nm) | 3.13E−06 | 3.51E−06 | 3.55E−06 | 3.36E−06 | 3.53E−06 | 3.83E−06 | W/cm² |
| UVB Irradiance (290-320 nm) | 3.01E−04 | 3.27E−04 | 3.17E−04 | 2.92E−04 | 3.28E−04 | 3.47E−04 | W/cm² |
| UVA Irradiance (320-400 nm) | 6.61E−03 | 7.04E−03 | 6.93E−03 | 6.37E−03 | 7.14E−03 | 7.32E−03 | W/cm² |
| UVA2 Irradiance (320-340 nm) | 8.15E−04 | 8.72E−04 | 8.53E−04 | 7.74E−04 | 8.69E−04 | 9.02E−04 | W/cm² |
| UVA1 Irradiance (340-400 nm) | 5.79E−03 | 6.17E−03 | 6.07E−03 | 5.60E−03 | 6.27E−03 | 6.42E−03 | W/cm² |
| Visible + NIR Irradiance | 5.95E−02 | 6.16E−02 | 5.94E−02 | 5.52E−02 | 6.36E−02 | 6.34E−02 | W/cm² |
| (400-800 nm) | | | | | | | |
| % UVC | 0.005% | 0.005% | 0.005% | 0.005% | 0.005% | 0.005% | |
| % UVB | 0.5% | 0.5% | 0.5% | 0.5% | 0.5% | 0.5% | |
| % UVA | 9.9% | 10.2% | 10.4% | 10.3% | 10.0% | 10.3% | |
| % Visible + NIR (400-800 nm) | 89.6% | 89.3% | 89.1% | 89.2% | 89.5% | 89.2% | |
| SED (seconds) | 194.6 | 175.6 | 178.9 | 193.5 | 175.8 | 163.6 | seconds |
| Erythemal Effective Irradiance | 5.14E−05 | 5.69E−05 | 5.59E−05 | 5.17E−05 | 5.69E−05 | 6.11E−05 | W/cm² |
| COLIPA In-vitro 2011 | | | | | | | |
| Total UV Irradiance | 69.8 | 74.4 | 73.1 | 67.3 | 75.4 | 77.3 | W/m² |
| 290-400 nm (50-140 W/m²) | | | | | | | Passed |
| Irradiance ratio of UVA320-400 nm | 21.95 | 21.56 | 21.85 | 21.83 | 21.77 | 21.09 | Passed |
| to UVB 290-320 nm (8-22) | | | | | | | |
| ISO 24443: 2012(E) | | | | | | | |
| Total UV Irradiance | 69.8 | 74.4 | 73.1 | 67.3 | 75.4 | 77.3 | W/m² |
| 290-400 nm (40-200 W/m²) | | | | | | | Passed |
| Irradiance ratio of UVA320-400 nm | 21.95 | 21.56 | 21.85 | 21.83 | 21.77 | 21.09 | Passed |
| to UVB 290-320 nm (8-22) | | | | | | | |

### Example 7: Fibroblast Cell Culture: Collagen, Hyaluronic Acid, Elastin

In this Example, a fibroblast cell culture model was used to assess the ability of a test material (i.e., Bacillus Lysate described herein) to exert an effect on type I collagen, hyaluronic acid, and elastin synthesis. This study also assessed the viability of the cells after exposure to the test materials.

### Summary of Test Method

Fibroblasts are the main source of extracellular matrix peptides, including structural proteins collagen and elastin. Procollagen is a large peptide synthesized by fibroblasts in the dermal layer of the skin and is the precursor for collagen. As the peptide is processed to form a mature collagen protein, the pro-peptide portion is cleaved off (type I C-peptide). Both the mature collagen protein and the type I C-peptide fragment are then released into the extracellular environment. As collagen is synthesized the type I C-peptide fragment accumulates into the tissue culture medium. Since there is a 1:1 stoichiometric ratio between the two parts of the procollagen peptide, assaying for type I C-peptide reflects the amount of collagen synthesized. Type 1 C-peptide is assayed via an ELISA based method.

Elastin is the main component of a network of elastic fibers that give tissues their ability to recoil after a transient stretch. This protein is released by fibroblasts (soluble elastin) into the extracellular space where it is then cross-linked to other elastin proteins to form an extensive network of fibers and sheets (insoluble elastin). Soluble elastin is measured from cell culture medium via an ELISA based method.

Changes in cell number are assessed via an MTT assay. The MTT assay is a colorimetric analysis of the metabolic activity of the cell, which is a reflection of the number of viable cells. Reduction of MTT by mitochondria results in the formation of insoluble purple formazin crystals that are extracted from the cells with isopropanol and quantified spectrophotometrically. The intensity of the purple color is directly proportional to the metabolic activity of the cells and inversely proportional to the toxicity of the test material.

### Methods

Preparation of Fibroblasts

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After this 24-hour wash out period the cells were treated with the test materials at the specified concentrations dissolved in FGM with 1.5% FBS. TGF-B (50 ng/ml) was used as a positive control for collagen and elastin, while 100 μM DbcAMP was used as a positive control for hyaluronic acid. Untreated cells (negative controls) just received DMEM with 1.5% FBS. The cells were incubated for 48 hours and at the end of the incubation period cell culture medium was collected and either stored frozen (−75° C.) or assayed immediately. Materials were tested in triplicate.

MTT Assay

After the 2-day incubation, the cell culture medium was removed (see above) and the fibroblasts were washed twice with PBS to remove any remaining test material. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts were transferred to a 96-well plate and the plate was read at 540 nm using isopropyl alcohol as a blank.

Type I Collagen Assay

A series of type I C-peptide standards was prepared ranging from 0 ng/ml to 640 ng/ml. Next, an ELISA microplate was prepared by removing any unneeded strips from the plate frame followed by the addition of 100 μl of peroxidase-labeled anti procollagen type I-C peptide antibody to each well used in the assay. Twenty (20) μl of either sample (collected tissue culture media) or standard was then added to appropriate wells and the microplate was covered and allowed to incubate for 3±0.25 hours at 37° C. After the incubation the wells were aspirated and washed three times with 400 μl of wash buffer. After the last wash was removed 100 μl of peroxidase substrate solution (hydrogen peroxide+tetramethylbenzidine as a chromagen) was added to each well and the plate was incubated for 15±5 minutes at room temperature. After the incubation 100 μl of stop solution (1 N sulfuric acid) was added to each well and the plate was read using a microplate reader at 450 nm.

Hyaluronic Acid Assay

A series of hyaluronic acid standards was prepared ranging from 50 ng/ml to 3,200 ng/ml. Next, 100 μl of each standard and sample was transferred to a well in an incubation plate. After adding 50 μl of detection solution to each well (except the reagent blank wells) the plate was incubated for 1±0.25 hour at 37±2° C. After the incubation, 100 μl of each sample/standard from the incubation plate was transferred to a corresponding well in the ELISA plate. The ELISA plate was covered and incubated for 30±5 minutes at 4° C. and then washed three times with 300 μl of wash buffer. After the final wash 100 μl of enzyme solution was added to each well and the plate was incubated at 37±2° C. for 30±5 minutes. After this incubation the wells were washed again as described above and then 100 μl of enzyme substrate solution was added to each well and the plate was incubated for 30-45 minutes at room temperature. After this final incubation 50 μl of stop solution was added to each well and the absorbance of the plate was measured at 405 nm using a plate reader.

Elastin Assay

A series of elastin standards was prepared ranging from 0 ng/ml to 300 ng/ml. Next, an ELISA microplate was prepared by removing any unneeded strips from the plate frame followed by the addition of 100 μl of each standard (prepared in duplicate) or sample. The microplate was then covered and allowed to incubate for one hour at 37° C. After the incubation the wells were decanted and 100 μl of detection reagent A was added and the plate was incubated again for one hour at 37° C. After this incubation the well plate was washed 3 times with wash solution (the plate was allowed to sit for 1-2 minutes at room temperature with each wash solution). After the final wash was decanted 100 μl of detection reagent B was added to each well and the plate was incubated for 30 minutes at 37° C. and then washed as described above. After the final wash was removed 90 μl of substrate solution was added to each well and the plate was incubated for 15±5 minutes at room temperature. After the incubation 50 μl of stop solution (1 N sulfuric acid) was added to each well and the plate was read using a microplate reader at 450 nm.

### Calculations

MTT Assay

The mean MTT absorbance value for the negative control cells was calculated and used to represent 100% cell viability. The individual MTT values from the cells undergoing the various treatments were then divided by the mean value for the negative control cells and expressed as a percent to determine the change in cell viability caused by each treatment.

ELISA Assays

To quantify the amount of each substance present, a standard curve was generated using known concentrations of each substance. A regression analysis was performed to establish the line that best fits these data points. Absorbance values for the test materials and untreated samples were used to estimate the amount of each substance present in each sample.

Statistical Analysis

Treatment means were compared using an ANOVA, with an n=3 per treatment. Statistical significance was set at p<0.05.

### Results

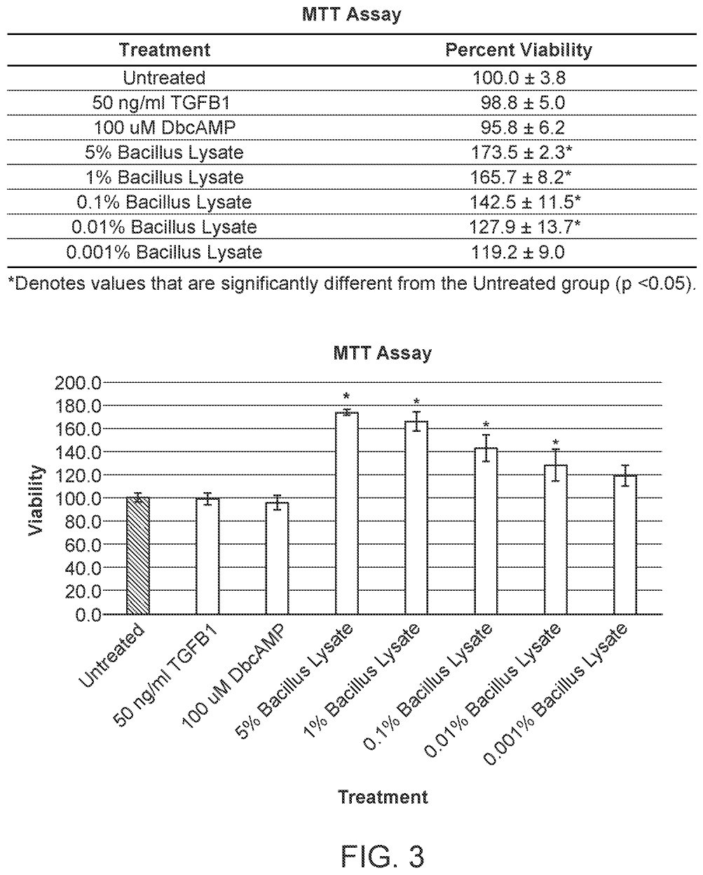

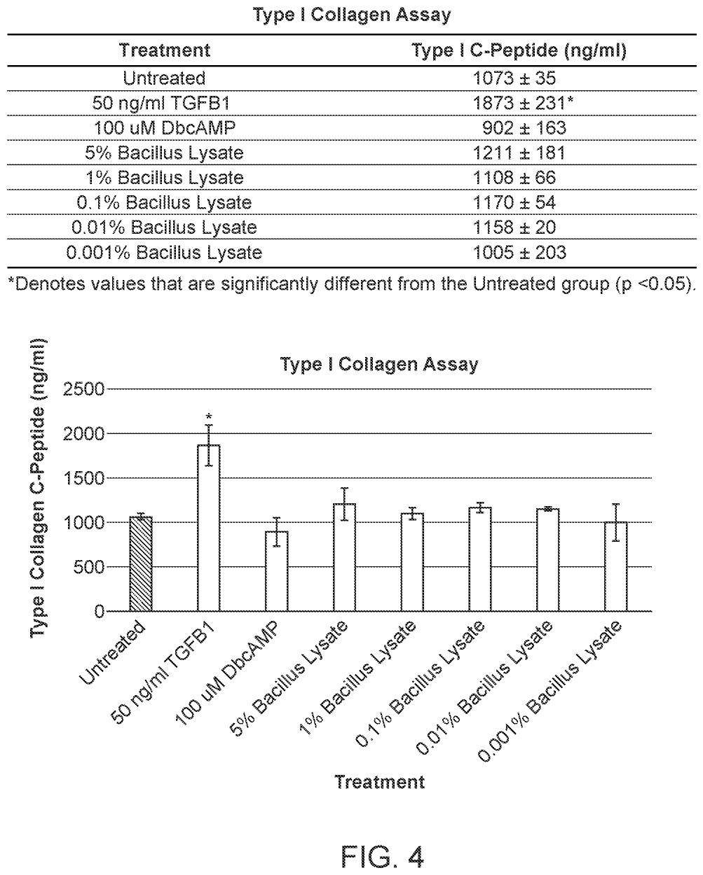

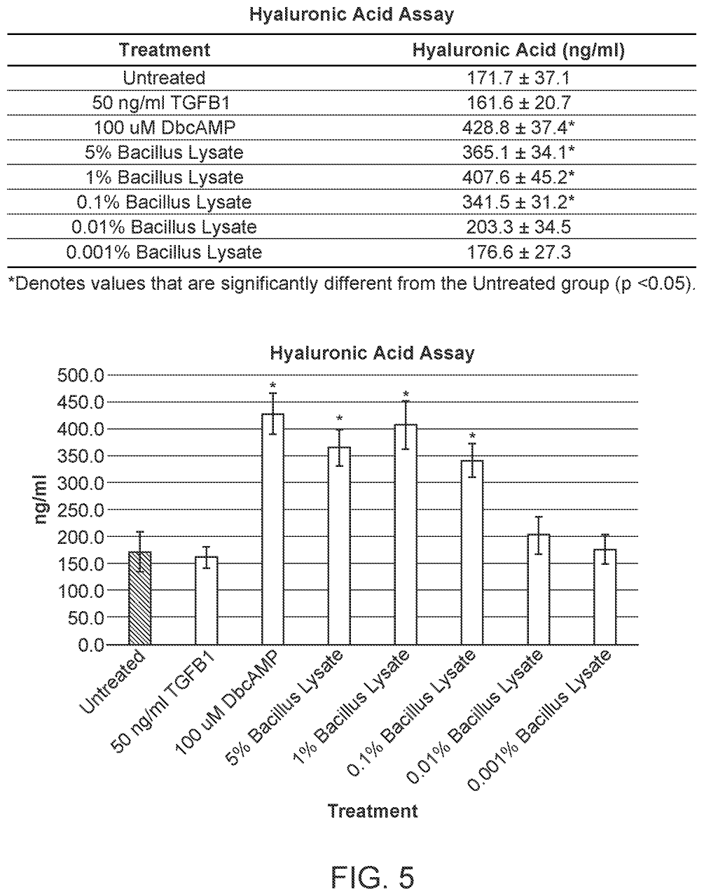

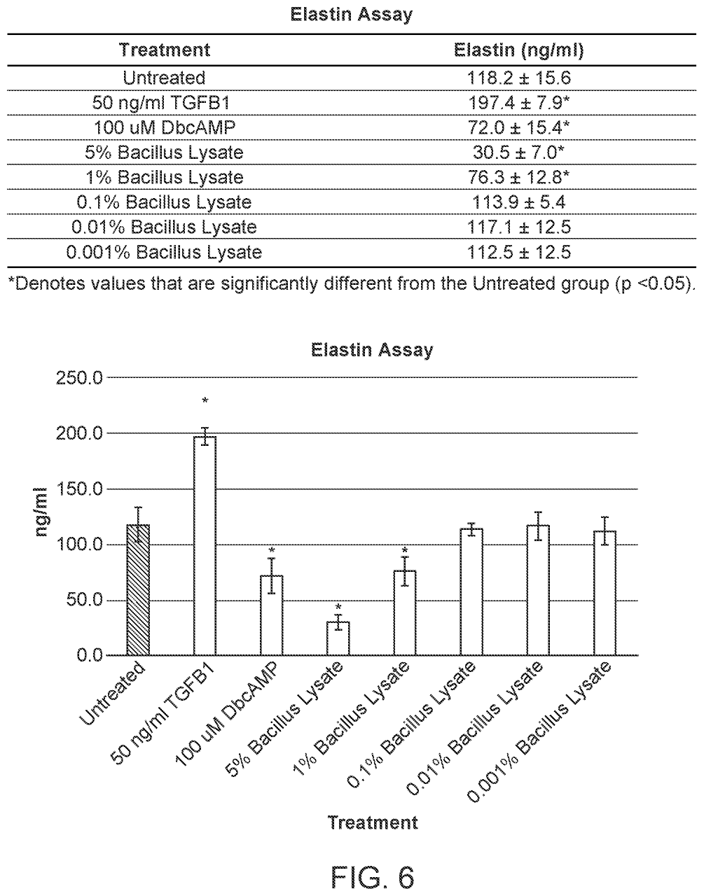

The results for the MTT assay are presented in FIG. 3, with the values presented as the mean percent viability±the standard deviation of the mean. The results for the ELISA assays are presented in FIG. 4 (Collagen), FIG. 5 (Hyaluronic Acid), and FIG. 6 (Elastin). These values are presented as mean concentration (ng/ml)±the standard deviation of the mean.

A fibroblast cell culture model was used to assess the ability of the test materials to exert an effect on type I collagen, hyaluronic acid, and elastin synthesis. The material was observed to significantly increase hyaluronic acid production and to decrease elastin production.

### Example 8: HORAC and ORAC Assay

In this Example, a Bacillus Lysate described herein was screened for antioxidant properties using non-cell-based methods.

### Summary of Test Methods

Fluorescein (FITC) is normally a highly fluorescent molecule, yet when fluorescein is oxidized by either peroxyl radicals or hydroxyl radicals it loses its fluorescence. Thus, when FITC is incubated in the presence of 2,2′-azobis(2-amidino-propane) dihydrochloride (AAPH, a peroxyl radical generating compound), or a combination of hydrogen peroxide and cobalt (a hydroxyl radical generating system based on the Fenton-type reaction), there is a time dependent loss of FITC fluorescence. However, if a material with antioxidant properties is present during the incubation, the loss of fluorescence is delayed as some of the peroxyl or hydroxyl radicals react with the antioxidant material instead of the FITC. Under these conditions, the delay or prevention in fluorescence decay is in proportion to the antioxidant capacity of the test material (i.e., a Bacillus Lysate described herein).

HORAC Assay (OxiSelect HORAC Assay, Cell Biolabs)

The HORAC assay was performed as specified by the manufacturer of the kit. For the assay, the test materials were prepared in Assay Buffer at 10× their final desired concentrations. Caffeic acid was used as the positive control for this assay. To start the assay, 20 μl of the test material (i.e., a Bacillus Lysate described herein) or positive control were added to the wells of a 96-well plate. All of the samples were prepared in triplicate. Next, 140 μl of an FITC solution was added to each well and the plate was incubated for 30 minutes in the dark. After this incubation, 20 μl of a hydrogen peroxide solution (Hydroxyl Radical Initiator) was added to each well, followed by the addition of 20 μl of a cobalt solution (Fenton Reagent). The plate was mixed and then read using a Fluoroskan Ascent Fluorometer at 3-minute intervals for 60 minutes with an excitation wavelength of 480 nm and an emission wavelength of 518 nm.

ORAC Assay

For the ORAC assay, the test materials (i.e., a Bacillus Lysate described herein) were prepared in Assay Buffer at 10× their final desired concentrations. Trolox was used as the positive control for this assay. To start the assay, 20 μl of the test material or positive control were added to the wells of a 96-well plate. All of the samples were prepared in triplicate. Next, 160 μl of a 360 nM FITC solution was added to each well and the plate was incubated for 30 minutes in the dark. After this incubation, 20 μl of a 153 mM AAPH (peroxyl radical generator) was added to each well. The plate was mixed and then read using a Fluoroskan Ascent Fluorometer at 2.5-minute intervals for 60 minutes with an excitation wavelength of 480 nm and an emission wavelength of 518 nm.

### Calculations

The loss of FITC fluorescence can be graphed by plotting fluorescence intensity vs time for each of the samples to generate a fluorescence decay curve. The area under this curve (AUC) then represents the extent of FITC fluorescence loss over the course of the assay, and this measurement is then used as an index for the effectiveness of the antioxidant samples. The AUC for a sample can be calculated using the following equation:

AUC=1+(RFU1/RFU0)+(RFU2/RFU0)+ . . . +(RFU final/RFU0)

Where RFU0 is the initial fluorescence measurement of the sample at time 0, with each subsequent RFU measurement indicated by the respective increased numbers. For this study, the AUC measurements were then used to determine the percent inhibition for each concentration of the test material screened, followed by the determination of the IC50, the concentration of the material at which 50% of the FITC signal loss due to radical interaction is prevented.

### Results

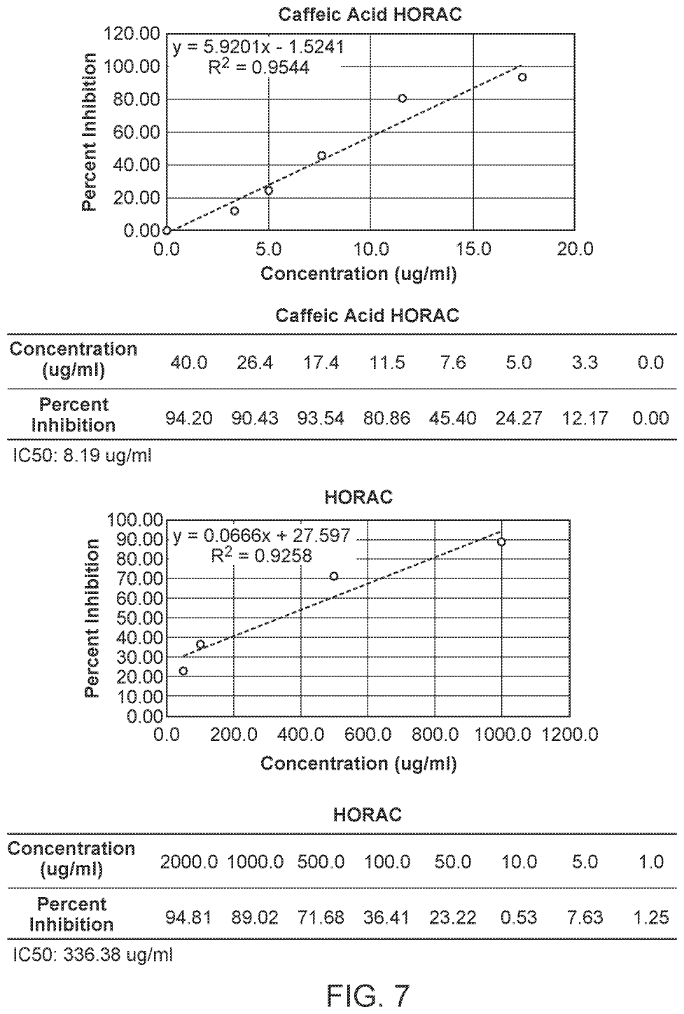

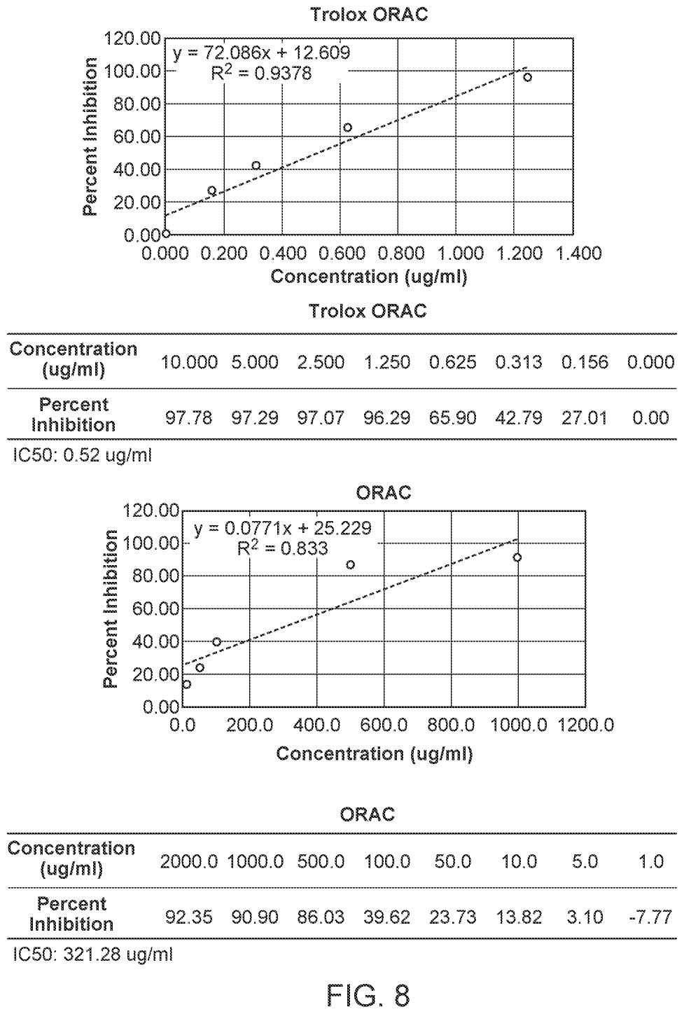

The results for the HORAC assay are presented in FIG. 7 (upper graph and upper table: positive control; lower graph and lower table: test material (i.e., Bacillus Lysate described herein)). The results for the ORAC assay are presented in in FIG. 8 (upper graph and upper table: positive control; lower graph and lower table: test material (i.e., Bacillus Lysate described herein)).

In this study, the test material (i.e., Bacillus Lysate described herein) was observed to scavenge both hydroxyl radicals (HORAC) and peroxyl radicals (ORAC). The IC50s for each of these were as follows:
HORAC: 336.38 μg/ml
ORAC: 321.28 μg/ml

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Certain implementations of the technology are set forth in the claims that follow.

What is claimed:

1. An isolated biologically purified culture of a strain of Bacillus pumilus deposited under ATCC accession number PTA-126909.

2. A composition comprising a strain of Bacillus pumilus deposited under ATCC accession number PTA-126909.

3. A composition comprising cellular remains from a strain of Bacillus pumilus deposited under ATCC accession number PTA-126909, wherein the composition is essentially free of viable cells of the strain of Bacillus pumilus.

4. The composition of claim 3, wherein the cellular remains are lysed, lyophilized from, microfluidized from or otherwise derived from the strain of Bacillus pumilus.

5. A composition comprising material secreted by cells of a strain of Bacillus pumilus deposited under ATCC accession number PTA-126909, wherein the secreted material is separated in whole or in part from the cells.

6. A composition comprising cellular remains and/or material secreted by cells of a strain of Bacillus pumilus deposited under ATCC accession number PTA-126909, wherein the composition is essentially free of viable cells of the strain of Bacillus pumilus.

7. A composition comprising cellular remains and/or material secreted by cells of the strain of Bacillus pumilus deposited under ATCC accession number PTA-126909, obtainable by a process comprising exposing cells of the strain of Bacillus pumilus deposited under ATCC accession number PTA-126909 to physical disruption that lyses bacterial cells.

8. The composition of claim 7, wherein the physical disruption comprises microfluidization, sonication and/or lyophilization.

9. The composition of claim 7 or claim 8, the process comprising concentrating the cellular remains and/or material secreted by the cells into an aqueous concentrate or pellet or supernatant in one or more steps, optionally comprising centrifugation.

10. The composition of any one of claims 7-9, comprising i) separating the cellular remains and/or material secreted by the cells from growth media, and ii) combining the cellular remains and/or material secreted by the cells with water to form an aqueous suspension and/or solution.

11. The composition of any one of claims 7-10, wherein the composition is essentially free of viable cells of the strain of Bacillus pumilus.

12. The composition of any one of claims 6-11, wherein the cellular remains and/or material secreted by the cells is dispersed uniformly in the composition at a weight percent of solids to liquids from 0.001% to 10%.

13. The composition of any one of claims 2-12, further comprising a liquid carrier.

14. The composition of any one of claims 2-13, wherein the composition is an additive for use in a consumer product selected from a cosmetic composition, a sunscreen and/or sunblock composition, and a topically-applied pharmaceutical composition.

15. The composition of any one of claims 2-13, wherein the composition is a consumer product selected from a cosmetic composition, a sunscreen and/or sunblock composition, and a topically-applied pharmaceutical composition.

16. The composition of claim 14 or claim 15, comprising one or more components chosen from an aqueous component, fatty component, volatile oil, non-volatile oil, surfactant, polymer, emulsifier, ultraviolet filter, sirtuin activator, anti-oxidant, and free-radical scavenger.

17. The composition of claim 16, wherein composition comprises an anti-oxidant chosen from vitamin E, Urolithin A, and combinations thereof.

18. The composition of claim 16, wherein the composition comprises a sirtuin activator, preferably resveratrol.

19. The composition of any one of claims 2-13, wherein the composition is an additive for use in a consumer product selected from paint, stain, lacquer, varnish, glaze, ink, a UV-stabilizer, a textile-treatment composition, a leather-treatment composition, a hair-treatment composition, and a fur-treatment composition.

20. The composition of any one of claims 2-13, wherein the composition is a consumer product selected from paint, stain, lacquer, varnish, glaze, ink, a UV-stabilizer, a textile-treatment composition, a leather-treatment composition, a hair-treatment composition, and a fur-treatment composition.

21. The composition of any one of claims 2-13, wherein the composition is a layered composition comprising (i) a surface layer of the strain of bacteria, its cellular remains, its secreted materials, or a mixture of its cellular remains and its secreted materials; and (ii) a substrate upon which the strain of bacteria, its cellular remains, its secreted materials, or a mixture of its cellular remains and its secreted materials are deposited as a surface layer.

22. The composition of any one of claims 2-12, wherein the composition is a solid composition comprising (i) an embedded amount of the strain of bacteria, its cellular remains, its secreted materials, or a mixture of its cellular remains and its secreted materials; and (ii) a solid material into which the strain of bacteria, its cellular remains, its secreted materials, or a mixture of its cellular remains and its secreted materials in embedded.

23. The composition of claim 22, wherein said composition is macroscopically homogeneous.

24. The composition of claim 22, wherein said composition is macroscopically heterogeneous.

25. A method providing protection from ultraviolet light comprising administering a composition according to claims 2-13 to a surface on a substrate or comprising integrating or embedding a composition according to claims 2-13 into a material.

26. The method of claim 25, wherein the substrate is alive, biologically-derived, or non-biological.

27. A method of making a composition according to claims 2-13 comprising obtaining bacteria of a strain of Bacillus pumilus deposited under ATCC accession number PTA-126909, optionally rendering the bacteria non-viable, optionally separating the bacteria from growth media, and adding the bacteria, or its cellular remains or secretions, or both its cellular remains and secretions, to a carrier to yield the composition.

28. A method of directed evolution to produce a bacteria with resistance to ultraviolet light, comprising obtaining a sample of Bacillus pumilus, growing the sample in artificial ultraviolet light at greater than or equal to 700 joules/m² of exposure (fluence), optionally approximately 900 joules/m² of exposure (fluence), and isolating viable bacteria surviving such growth conditions, wherein the isolated viable bacteria exhibit greater UV absorption compared to the original Bacillus pumilus from which the isolated viable bacteria was derived, optionally wherein the greater UV absorption is measured by comparison of UV absorption of lyophilized powders from the original Bacillus pumilus and the subsequent isolated viable bacteria, in each case re-suspended in diH₂O at a concentration ranging from 0.1% w:v to 0.5% w:v.

29. A composition comprising bacteria, or cellular remains, secretions, or both cellular remains and secretions, of such bacteria, wherein the bacteria are produced according to the method of claim 28.

30. A composition, comprising cellular remains of bacteria, or secretions of bacteria, or cellular remains and secretions of bacteria, wherein the composition is (i) a consumer product selected from a cosmetic composition, a sunscreen and/or sunblock composition, a sun protection factor (SPF) booster, and a topically-applied pharmaceutical composition, or (ii) an additive for use in a consumer product selected from a cosmetic composition, a sunscreen and/or sunblock composition, a sun protection factor (SPF) booster, and a topically-applied pharmaceutical composition, wherein said bacteria have been artificially selected for UV-resistance.

31. The composition of claim 30, obtainable by a process comprising growing the bacteria in artificial ultraviolet light and thereafter exposing the bacteria to mechanical disruption.

32. The composition of claim 31, wherein the physical disruption comprises microfluidization, sonication and/or lyophilization.

33. The composition of claim 31 or claim 32, the process comprising concentrating the cellular remains and/or secretions into an aqueous concentrate or pellet or supernatant.

34. The composition of any one of claims 31-33, comprising combining the cellular remains and/or secretions with water to form an aqueous suspension and/or solution at a weight percent of solids to liquids from 0.001% to 10%.

35. The composition of any one of claims 31-34, wherein the composition is essentially free of viable cells of the bacteria and essentially free of growth media.

36. The composition of any one of claims 31-35, wherein the artificial ultraviolet light is greater than or equal to 700 joules/m2 of exposure (fluence), and optionally approximately 900 joules/m2 of exposure (fluence).

37. The composition of any one of claims 31-36, wherein the process comprises isolating the bacteria after growing in the artificial ultraviolet light prior to exposing the bacterial to mechanical disruption.

38. The composition of any one of claims 31-37, wherein the bacteria were exposed to ultraviolet light naturally occurring in the stratosphere of Earth or beyond the stratosphere of Earth prior to growing the bacteria in the artificial ultraviolet light.

39. The composition of claim 38, wherein the bacteria were exposed to the ultraviolet light naturally occurring in the stratosphere of Earth or beyond the stratosphere of Earth for about 18 months or more.

40. The composition of any one of claims 30-39, wherein the bacteria are Bacillus bacteria.

41. The composition of claim 40, wherein the bacteria are Bacillus pumilus bacteria.

42. The composition of claim 41, wherein the bacteria are Bacillus pumilus bacteria deposited under ATCC accession number PTA-126909.

43. The composition of any one of claims 30-42, wherein the bacteria is substantially homogeneous.

44. The composition of any one of claims 30-43, comprising one or more components chosen from an aqueous component, fatty component, volatile oil, non-volatile oil, surfactant, polymer, emulsifier, ultraviolet filter, sirtuin activator, anti-oxidant, and free-radical scavenger.

45. The composition of claim 44, wherein the composition comprises an anti-oxidant chosen from vitamin E, Urolithin A, and combinations thereof.

46. The composition of claim 44, wherein the composition comprises a sirtuin activator, preferably resveratrol.

47. The composition of any one of claims 30-43, wherein the cellular remains and/or secretions of bacteria is/are about 5% or less by weight in the composition.

48. The composition of any one of claims 30-46, which is stable for 24 hours or longer.

49. The composition of any one of claims 30-48, comprising xanthan gum in an amount of greater than zero to about 2% by weight, preferably up to about 0.2% by weight or less.

50. The composition of any one of claims 30-49, which blocks blue spectrum ultraviolet light relative to an otherwise identical composition not comprising the cellular remains of the bacteria and/or secretions of the bacteria.

51. The composition of any one of claims 30-50, for increasing an amount of skin hyaluronic acid.

52. A method for increasing an amount of skin hyaluronic acid for a subject, comprising administering topically a composition of any one of claims 30-50 to skin of a subject in an amount effective to increase the amount of skin hyaluronic acid.

53. A method for boosting sun protection factor (SPF) in a composition, comprising: combining a composition of any one of claims 30-50 with a base composition chosen from a cosmetic composition, a sunscreen and/or sunblock composition, and a topically-applied pharmaceutical composition, thereby providing a combined composition, wherein:
the base composition has a first SPF value prior to the combining, and
the combined composition has a second SPF value greater than the first SPF value.

54. A method for protection of skin from ultraviolet light, comprising administering a composition of any one of claims 30-50 to skin of a subject.

55. The composition of any one of claims 30-50, for protecting skin from HEV light.

56. A method for protection of a surface and/or skin of a subject from HEV light, comprising administering a composition of any one of claims 30-50 to said surface and/or skin of a subject.
